(19) United States
(12) Patent Application Publication
(10) Pub. No.: US 2024/0245830 A1
(43) Pub. Date: Jul. 25, 2024

(54) MULTI-LAYER AMNIOTIC TISSUE GRAFTS AND USES THEREOF

(71) Applicant: Celularity Inc., Florham Park, NJ (US)

(72) Inventors: Adam KUEHN, Short Hills, NJ (US); Desiree LONG, Palmer, PA (US); Rajarajeswari SIVALENKA, Avenel, NJ (US); Anna GOSIEWSKA, Skillman, NJ (US); Stephen A. BRIGIDO, Bethlehem, PA (US); Timothy F. WILK, Kinnelon, NJ (US); Amanda L. TRINKA, Hampton, NJ (US); Luis MARTINEZ, Milltown, NJ (US); Robert J. HARIRI, Bernardsville, NJ (US)

(73) Assignee: Celularity Inc., Florham Park, NJ (US)

(21) Appl. No.: 18/555,412

(22) PCT Filed: Apr. 13, 2022
(86) PCT No.: PCT/US2022/071705

§ 371 Date: Oct. 13, 2023

Related U.S. Application Data
(60) Provisional application No. 63/174,280, filed on Apr. 13, 2021.
(60) Provisional application No. 63/267,820, filed on Feb. 10, 2022.

(51) Int. Cl. A61L 27/36

(52) U.S. Cl. CPC A61L 27/3633; A61L 27/3641; A61L 2430/16

(57) ABSTRACT

The present invention provides a tissue graft product comprising a plurality of laminated layers of extracellular matrix. wherein the extracellular matrix is derived from an amniotic membrane, and wherein the stromal side of an extracellular matrix layer is presented on both the upper and lower surfaces of the tissue graft product. Methods of making and using the tissue graft product also are provided.

This application claims priority to U.S. Provisional Patent Application Nos. 63/174,280, filed Apr. 13, 2021, and 63/267,820, filed Feb. 10, 2022, the contents of which are incorporated herein by reference in their entireties.

### FIELD

The present invention relates, in part, to multi-layered amniotic tissue grafts and their use in oclar applications.

### BACKGROUND

Human amniotic membrane (amnion) is the innermost layer of the amniotic sac which comes in direct contact with amnion fluid. It consists of a single layer of cuboidal epithelial cells, a basement membrane, and an avascular stromal matrix loosely attached to the chorion. The major components of human amnion are reported to be collagen and elastin. Other biochemical component, such as laminins and proteoglycans, also present in small quantities.

BIOVANCE is manufactured from human amniotic membrane. The raw-material amniotic membrane undergoes rinse and decellularization processes which are designed to clean the blood component contaminantion and remove cells from the membrane without altering the native collagen-based architecture. The cleaned and decellularized amniontic membrane is dehydrated at a mild temperature of 50 oC so that the final product will be easy to store, transport and have longer shelf life. The product is terminally sterilized using e-beam radiation.

Amniotic membrane (AM) is used for ocular surface reconstruction to treat a variety of ocular pathologies, including corneal surface disorders with and without limbal stem cell deficiency, as a carrier for ex vivo expansion of limbal epithelial cells, conjunctival surface reconstruction, (e.g., pterygium removal, after removal of large lesions other than pterygium, after symblepharon lysis), glaucoma, neoplasia, pterygium, as well as sclera melts and perforations (Walkden, 2020; Elhassan, 2019; Malhotra & Jain, 2014, Mamede et al. 2012).

AM can be used as either a patch or a graft. By placing the AM epithelial cell up, the AM acts as a substrate and scaffold for epithelial cell growth (Malhotra & Jain, 2014). As a patch, the AM acts as a temporary biological bandage or contact lens, promoting re-epithelization of the host tissue beneath the patch (Walden, 2020, Malhotra & Jain, 2014). Placing the AM as a patch stromal side down is thought to downregulate the inflammatory response by trapping inflammatory cells and inducing apoptosis (Dua et al. 2004). Therefore, AM is placed stromal side down in the presence of acute inflammation, especially when associated with epithelial defects, to protect the ocular surface from inflammatory cells and mediators (Malhotra & Jain, 2014; Mamede et al. 2012).

TABLE 1
Uses of AM as a graft or patch based on the ocular
pathology (Safa Elhassan, 2019;
Understanding Amniotic Membrane Grafts).

| | Graft | Patch |
|---|---|---|
| Cornea | Small perforations | Persistent epithelial defects; |
| | or melts, secondary to | Neurotrophic keratitis; Band |
| | corneal ulcers or | keratopathy; Bullous |
| | thinning (sterile) | keratopathy; Limbal stem |
| | | cell disease; recurrent |
| | | epithelial erosion; corneal |
| | | dystrophy |
| Conjunctiva | Pterygium excision; | Symblepharon; Mechanical |
| | Bleb reconstruction; | trauma; Chemical trauma; |
| | Symblepharon; | Stevens-Johnson syndrome |
| | reconstruction of the | |
| | conjunctiva and fornix, | |
| | following tumour | |
| | excision or cicatrizing | |
| | disease | |
| Scleral | Small scleral | Large melts or perforations |
| | perforations and melts | |
| Others | Eyelid reconstruction | Glaucoma or cataract surgery; |
| | | Dysfunctional tear syndrome |

AM Orientation & Application Methods: Selection of the application method is dependent upon the indication(s) for use, the desired outcome, and the depth and size of the wound (Walkden, 2020; Elhassan, 2019; Malhotra & Jain, 2014).

Three application methods are consistently reported throughout the literature:
Inlay Technique (Permanent Graft):
Onlay Technique (Temporary Biological Bandage or Contact Lens); and
Combined Inlay-Onlay Technique (Permanent Graft & Temporary Biological Bandage).

Inlay Technique (Permanent Graft): The AM is placed epithelial/basement membrane side up to provide the host's cells a substrate on which they can grow. Over time, the AM matrix is remodeled into the host cornea. Hence, it is serving as a permanent graft.

The AM is trimmed to fit the defect, placed epithelial side up, and is usually sutured to the cornea. Approximately, 2 mm of the host's corneal epithelium is debrided. This allows the regenerating epithelium to grow over the epithelial/basement membrane of the AM. Depending on the size of the defect, a single or multilayer technique can be used. With the multilayer technique, the AM can be cut into several pieces or blanket folded.

Onlay Technique (Temporary Biological Bandage or Contact Lens): The AM can be placed either epithelial/basement membrane side up or stromal side up because the host's epithelium is intended to grow under the membrane. The AM is expected to fall off, be removed, or self-degrade over a period of time. Therefore, the AM is serving as temporary biological bandage or contact lens, providing a physical barrier. It is not intended to be incorporated into host tissue.

The AM is sized larger than the defect, so that there is host epithelium present beneath the membrane. It is either sutured or glued in place.

Combined Inlay-Onlay Technique: This combines both the inlay and the onlay techniques. As described above, AM is placed epithelial side up in the defect and is expected to incorporate into the host tissue. Either a single layer or multilayer technique can be used. This is combined with the onlay technique where the graft is placed either epithelial/basement membrane side up or stromal side up, extending beyond the perimeter of the defect. With this technique, the epithelium is expected to grow under the patch but over the uppermost inlay graft.

### SUMMARY

The present invention provides tissue graft products comprising a plurality of layers of extracellular matrix laminated together, wherein the extracellular matrix is derived from an amniotic membrane, and wherein the stromal side of an extracellular matrix layer is presented on both the upper and lower surfaces of the tissue graft product.

The present invention also provides ocular tissue grafts comprising tissue graft products of the invention.

The present invention also provides methods of treating a disease or injury of the eye in a subject, the method comprising the step of contacting the eye of the subject with tissue graft products or the ocular tissue grafts of the invention.

### BRIEF DESCRIPTION OF THE FIGURES

FIG. 1 shows cell adhesion by side and amniotic membrane. Means and standard deviations are plotted. Cell adhesion measured in fluorescent intensity (AU).

FIG. 2 shows cell proliferation. Means and standard deviations are plotted.

FIG. 3 shows crelative proliferation rate. Means and standard deviations are plotted.

FIG. 4 shows migration area by Amniotic Membrane. Means and standard deviations are plotted. Migration area is reported as px².

FIG. 5 shows cell viability on the E&S sides of AMs over 7 days. * p≤0.05, compared with DDHAM-S.

FIG. 6 shows that after 4 days, cells on the S side of AMs were stained with CalceinAM to visualize viable cells (A) and with phalloidin to visualize actin (B).

FIG. 7 shows gene expression of TNFa in HCECs cultured on AMs for 24 h, 48 h and 72 h. *p≤0.05.

FIGS. 8A and 8B show immunofluorescent and H&E staining of amniotic membranes. Immunofluorescent staining of DDHAM, DHAM, and CHAM is shown (A). The cross-sections of the membranes were stained with Hoechst Dye (DNA in blue), phalloidin (Actin in green) and anti-human type I collagen antibodies (Coll in red). Representative images are shown and the scale bar=50 um. H&E staining (nuclei in blue and cytoplasm in red) of DDHAM, DHAM, and CHAM is shown (B). Representative images are shown and the scale bar=20 um.

FIG. 9 shows cell adhesion. Human corneal epithelial cells seeded onto the epithelial and stromal sides of amniotic membranes and incubated for 24 h. Comparisons between the epithelial and stromal sides of each amniotic membrane are shown, and comparisons between amniotic membranes for each side are shown. Means and standard deviations are plotted. Fluorescent intensity is expressed in arbitrary units (AU). Data shown are mean±SD. *p≤0.05. Abbreviations: CHAM, cryopreserved human amniotic membrane: DDHAM, decellularized dehydrated human amniotic membrane: DHAM, dehydrated human amniotic membrane.

FIG. 10 shows Staining of human corneal epithelial cells on AMs at day 4. Human corneal epithelial cells were seeded onto the stromal side of the three AMs, cultured, and stained with Calcein AM to visualize viable cells at Day 4 (A). The morphology of human comeal epithelial cells on AMs was monitored by actin staining on Day 4 and pseudo-colored red (B). Images were captured using epi-fluorescent microscope. Scale bar=100 μm. Abbreviations: CHAM, cryopreserved human amniotic membrane; DDHAM, decellularized dehydrated human amniotic membrane: DHAM, dehydrated human amniotic membrane.

FIGS. 11A and 11B show cell viability over time. Human corneal epithelial cells were seeded onto the epithelial and stromal sides of amniotic membranes and incubated for 1, 4 and 7 days. The viabilities of cells on amniotic membranes were measured by alamarBlue assay at each time point. Fluorescent intensity is expressed in arbitrary units (AU). Means and standard deviations are plotted over time for each side of the amniotic membranes (A). The relative cell viability, expressed as a percentage of day 1, and standard deviations are plotted across time for each of the amniotic membranes. Comparisons between the epithelial and stromal sides of each amniotic membrane are shown and comparisons between the amniotic membranes for each side are shown. Data shown are mean±SD. *p≤0.05. Abbreviations: CHAM, cryopreserved human amniotic membrane: DDHAM, decellularized dehydrated human amniotic membrane: DHAM, dehydrated human amniotic membrane.

FIGS. 12A and 12B show quantification of migration. Representative scratch wound images are shown to demonstrate the effects of conditioned media on the migration of human corneal epithelial cells at 0 h and 24 h (A). The conditioned media from different amniotic membranes (with and without cells) were tested to evaluate the effect of AMs alone on the migration of human corneal epithelial cells (B). The wound areas were measured using Image J and expressed in square pixels (px2). The migrated area=Area0 h−Area24 h. Data shown are mean±SD. *p≤0.05. Abbreviations: CHAM, cryopreserved human amniotic membrane: DDHAM, decellularized dehydrated human amniotic membrane: DHAM, dehydrated human amniotic membrane: Medium Ctrl, control.

FIGS. 13A-13D show mRNA Expression at 24 hours. Relative mRNA expression of GM-CSF (A), IL-6 (B), IL-8 (C), and TNF-α (D) at 24 hours are shown. Relative mRNA expression at 24 hours is normalized to TCP in the resting condition. Data shown are mean±SD. *p≤0.05. Abbreviations: CHAM, cryopreserved human amniotic membrane: DDHAM, decellularized dehydrated human amniotic membrane; DHAM, dehydrated human amniotic membrane: GM-CSF, granulocyte macrophage colony-stimulating factor: IL-6, interleukin-6; IL-8, interleukin-8: TNF-α, tumor necrosis factor alpha.

FIGS. 14A-14D show mRNA expression across time. Relative mRNA expression of GM-CSF (A), IL-6 (B), IL-8 (C), and TNF-α (D) across time in the stimulated condition (+TNF-α) are shown. Relative mRNA expression across time is normalized to expression at 24 hours. Statistical comparisons are between time points for each amniotic membrane in the stimulated condition. Data shown are mean±SD. *p≤0.05. Abbreviations: CHAM, cryopreserved human amniotic membrane: DDHAM, decellularized dehydrated human amniotic membrane: DHAM, dehydrated human amniotic membrane; GM-CSF, granulocyte macrophage colony-stimulating factor: IL-6, interleukin-6; IL-8, interleukin-8: TNF-α, tumor necrosis factor alpha.

FIGS. 15A-15F show a clinical case study. Images of the epithelial surface were taken to illustrate the clinical course: pre-operatively, showing the poor irregular surface of the epithelium (A), post removal of poor epithelium with visible sub epithelial debris from Anterior Basement Membrane Dystrophy (B), post burring of all sub-epithelial scarring and Anterior Basement Membrane Dystrophy debris (C), placement of DDHAM (D), placement of bandage contact lens over DDHAM (E), and one month postoperatively, showing a clear surface.

FIGS. 16A-16C show ocular AM preparation. DDHAM is packaged as 10 mm discs (A). For the study, DHAM (B) and CHAM (C) were made into 10 mm discs using a 10 mm biopsy punch. Abbreviations: CHAM, cryopreserved human amniotic membrane: DDHAM, decellularized dehydrated human amniotic membrane: DHAM, dehydrated human amniotic membrane.

FIG. 17 shows models of 3D printed molds allowing Biovance 3L ocular to be dried into curved shapes.

FIG. 18 shows Biovance 3L ocular which has been dried into curved shapes.

### DETAILED DESCRIPPTION

The present invention provides tissue graft products comprising a plurality of layers of extracellular matrix laminated together, wherein the extracellular matrix is derived from an amniotic membrane, and wherein the stromal side of an extracellular matrix layer is presented on both the upper and lower surfaces of the tissue graft product.

In some embodiments, the product comprises three or more layers of extracellular matrix. In some embodiments, product comprises exactly three layers of extracellular matrix.

In some embodiments, the amniotic membrane is decellularized. In some embodiments, the amniotic membrane is decellularized with a detergent and or mechanical disruption. In some embodiments, the detergent is deoxycholic acid.

In some embodiments, the plurality of layers of extracellular matrix are laminated together by drying. In some embodiments, the product is dried by heat and or vacuum.

In some embodiments, the tissue graft product is dehydrated. In some embodiments, the product comprises less than about 20% water by dry weight. In some embodiments, the product comprises less than about 15% water by dry weight. In some embodiments, the product comprises about 10% water by dry weight.

In some embodiments, the product comprises about 40% to about 70% total collagen by dry weight. In some embodiments, the product comprises about 45% to about 60% total collagen by dry weight. In some embodiments, the product comprises about 50% to about 55% total collagen by dry weight. In some embodiments, the collagen is primarily collagen type I and collagen type III.

In some embodiments, the product comprises about 8% to about 24% elastin by dry weight. In some embodiments, the product comprises about 12% to about 20% elastin by dry weight. In some embodiments, the product comprises about 15% to about 20% elastin by dry weight.

In some embodiments, the product comprises less than about 1% glycosaminoglycan by dry weight. In preferred embodiments, the product comprises less than about 0.5% glycosaminoglycan by dry weight. In some embodiments, the product comprises less than about 1% fibronectin by dry weight. In preferred embodiments, the product comprises less than about 0.5% fibronectin by dry weight. In some embodiments, the product comprises less than about 1% laminin by dry weight. In preferred embodiments, the product comprises less than about 0.5% laminin by dry weight.

In some embodiments, the amniotic membrane is a human amniotic membrane. In some embodiments, the amniotic membrane is derived from a full-term pregnancy.

The present invention also provides ocular tissue grafts comprising tissue graft products of the invention.

In some embodiments, the ocular tissue graft is approximately circular. In some embodiments, the ocular tissue graft comprises a curved portion in the shape of a portion of a sphere.

In some embodiments, the shape is imparted by drying the tissue graft product onto a mold.

The present invention also provides methods of treating a disease or injury of the eye in a subject, the method comprising the step of contacting the eye of the subject with tissue graft products or the ocular tissue grafts of the invention.

In some embodiments, the injury of the eye comprises an abrasion. In some embodiments, the injury of the eye comprises a chemical exposure. In some embodiments, the injury of the eye comprises a cut or laceration. In some embodiments, the disease or injury of the eye comprises a disease or injury of the cornea.

In some embodiments, the treatment comprises repair of a damaged tissue. In some embodiments, the treatment comprises reduction in scar tissue or reduction in scar tissue formation relative to an untreated eye. In some embodiments, the treatment comprises increased epithelial cell migration relative to an untreated eye. In some embodiments, the treatment comprises increased epithelial cell adhesion relative to an untreated eye. In some embodiments, the treatment comprises increased epithelial cell proliferation relative to an untreated eye. In some embodiments, the treatment comprises increased epithelial cell coverage relative to an untreated eye.

In some embodiments, the subject is a mammal. In preferred embodiments, the subject is a human.

### EXAMPLES

### Exaple 1

### Biochemical Composition of Biovance

BIOVANCE is composed primarily of collagen and elastin. Glycosaminoglycan, fibronectin, and laminin are also present in small amounts.

TABLE 2
Biochemical Composition.

| | MEAN VALUE | RANGE |
|---|---|---|
| | (% TOTAL WEIGHT | (% TOTAL |
| | OF MEMBRANE) | WEIGHT OF |
| TEST PARAMETER | N = 15 (3 PER LOT) | MEMBRANE) |
| TOTAL COLLAGEN | 52.9 | 40.7-66.2 |
| ELASTIN | 18.9 | 10.6-23.2 |
| GLYCOSAMINO- | 0.3 | 0.19-0.33 |
| GLYCAN | | |
| FIBRONECTIN | 0.3 | 0.12-0.73 |
| LAMININ | 0.1 | 0.01-0.16 |
| OTHER PROTEINS | 17 | |

* Proteins listed as “Other” include, but not limited to, collagen Types V, VI and VII, integrin, and fibronectin precursor.

TABLE 3
Collagen Sub-Type Composition

| | MEAN VALUE | |
|---|---|---|
| | (% TOTAL WEIGHT | (% TOTAL |
| | OF MEMBRANE) | WEIGHT OF |
| TEST PARAMETER | N = 15 (3 PER LOT) | MEMBRANE) |
| COLLAGEN TYPE I | 23.2 | 13-33 |
| COLLAGEN TYPE III | 28.9 | 20-44 |
| COLLAGEN TYPE IV | 0.8 | 0.5-1.2 |

### Example 2

### A Comparison Study of the Effects of Ocular Scaffolds on Human Ocular Epithelial Cells

Amniotic scaffolds due to their unique biological properties have been used for the treatment of various ocular diseases. Bench top data has demonstrated that the regenerative properties of scaffolds can impact innate healing mechanisms. Amniotic scaffolds can help to speed natural healing and reduce subjective pain and surgical complications. Despite extensive research documenting the inherent regenerative capacity of amniotic scaffolds, the acquisition and processing of the tissue is constantly evolving. Additional efforts are needed to elucidate which processing methodology produces a scaffold ideal for Ophthalmic application.

Purpose: To determine the effect of three amniotic scaffolds (Biovance3L Ocular, AMBIO2®, AmnioGraft®) on human ocular epithelial cell adhesion and proliferation.

Methods: Human corneal epithelial cells (HCEC) and human conjunctival epithelial cells (HConEpiC) were seeded into wells. Adhesion and proliferation were measured at days 1, 4 and 7 on the scaffolds. Conditioned media were extracted from wells and used for growth assays.

Results: Compared with the two other scaffolds, Biovance3L Ocular showed significantly higher epithelial cell viability (P<0.001) and demonstrated significantly greater epithelial cell adhesion (P≤0.011). Moreover, the rate of epithelial cell proliferation was significantly greater on Biovance3L than AmnioGraft® (P<0.001). HCEC migration in the presence of conditioned media from cells cultured on Biovance3L Ocular and AMBIO2® were comparable (P=0.885) and significantly greater than cells grown on AmnioGraft® (P≤0.006). The migration of HCEC in the presence of conditioned media from cells cultured on ocular scaffolds was significantly greater than control conditioned media from cells grown on tissue culture plastic (P<0.001). The conditioned media from different scaffolds did not affect the migration of HConEpiC.

Conclusions: Biovance3L Ocular had a significant effect on human epithelial cells by supporting greater viability, adhesion, and proliferation of both HCEC and HConEpiC when compared with other scaffolds. Additional research is needed to assess the clinical impact of these findings.

Summary: Biovance3L Ocular was compared against market competitors AMBIO2® and AmnioGraftR to determine cellular growth differences in various assays. Biovance3L demonstrated superior viability, adhesion, and proliferation of HCEC and HConEpiC when compared with other scaffolds. Biovance3L Ocular, which is devoid of residual cells, DNA, growth factors and cytokines, demonstrated superior growth measurements for ocular epithelial cells, which is critical to achieving natural repair and regeneration.

### Example 3

### Biovance 3L

Background: Amniotic membranes have broad clinical uses. Typically, a single layer membrane is utilized across clinical applications.

It has been documented that the preferred orientation for corneal epithelial cell re-epithelialization is the epithelial side of the amniotic membrane, supporting re-epithelialization, as compared to the stromal side.

For example, D. J Hu (Investigative Ophthalmology & Visual Science May 2003, Vol. 44. 3151), has compared corneal re-epithelialization over amniotic membrane (AM) sutured on corneal defects in two orientations: AM basement membrane anterior (BMA) and AM basement membrane posterior (BMP) side. His conclusion was that corneal re-epithelialization rates are not influenced by the orientation of the AM. Corneal epithelium has a greater affinity toward the basement membrane (epithelial side) of the AM, regardless of orientation. Clinicians should consider this finding and realize that while epithelium may grow on both sides of the amniotic membrane, the majority of re-epithelialization takes place on the basement membrane surface.

Our Research Question/Hypothesis: How does the sidedness (i.e., epithelial, stromal) and different membrane processing methodologies (i.e., DDHAM, DHAM, and CHAM) of amniotic membranes (AM) influence the adhesion, proliferation, and migration of HCECs?

In addition, our hypothesis is that our proprietary decellularization process, aiming at complete removal of residual cellular components, cells, cell debris, DNA, growth factors, and cytokines, as well as retention of an intact innate collagen framework with essential extracellular matrix molecules, in a native 3-dimensional form, provides superior biocompatibility and the ability to support cell differentiated functions, as compared with other amnion—derived products, containing residual cells, cell debris, DNA and growth factors and cytokines.

We conceived a 3 -layered membrane, called 3L, which differs from single layered membranes, in those three layers, instead of one are dried together, forming a new material configuration. The novel layering step was added before the membrane drying process creating a product with unexpected new properties and clinical uses. As part of this novel composition, the amnion is layered so that it is three layers thick with the stromal side outward on the top and the bottom. This membrane is layered onto itself and dried to create a membrane of three layers from the same amniotic membrane.

Our new product consists of amniotic membrane that is stripped from the placenta and placed into a mild detergent, 1% Deoxycholic acid, for soaking. The amnion membrane undergoes mechanical scraping intended to remove nearly 100% of the amniocytes and chorionic cells from the surface of the membrane and the vast majority of the fibroblasts from the substance of the tissue.

The final product is a tri-layer amniotic membrane structural tissue composed of extracellular matrix that retains the native collagen structure of amniotic membrane, including elastin and fibronectin that binds to collagen and other matrix components.

The novel layering step was added before the membrane drying process creating a product with unexpected new properties and clinical uses.

As part of this novel composition, the amnion is layered so that it is three layers thick with the stromal side outward on the top and the bottom. Once dry the amnion is cut into the desired sizes, each individual piece is placed into an inner pouch, labeled, sealed, and sterilized.

Biovance 3L unexpected results are related to differences in human comeal epithelial and human conjunctival cell attachment, proliferation, and migration on the stromal versus epithelial side of 3L BIOVANCE Ocular. In addition, the decellularization process has an impact on amniotic membrane performance.

Statistical Analysis: The independent variables are AM (DDHAM, DHAM, CHAM), side (epithelial, stromal), and time (day 1, day 4, and day 7). The dependent variables are cell adhesion, proliferation, and migration. The following results are for human corneal epithelial cells on both the epithelial and stromal sides of three amniotic membranes (AM): Biovance3L Ocular (DDHAM), AMBIO2 (DHAM), and AmnioGraft (CHAM).

Data are shown as mean±standard deviation (SD). The data were tested and found to be approximately normally distributed. Cell adhesion and migration were analyzed with a two-way analysis of variance (ANOVA) with Tukey post-hoc tests. Cell proliferation was analyzed with a three-way ANOVA with Tukey post-hoc tests. ANOVA results are reported as an F-statistic and its associated degrees of freedom. Unpaired t-tests were conducted as post-hoc tests when indicated. A p-value<0.05 was considered significant. All analyses were conducted using IBM SPSS (Build 1.0.0.1444).

Biovance® Tri-layer is a tri-layered, decellularized, dehydrated human amniotic membrane (DDHAM) with a preserved natural epithelial basement membrane and an intact extracellular matrix structure with its biochemical components. The epithelial basement membrane and extracellular matrix of this allograft provide a natural scaffold that allows cellular attachment or infiltration and growth factor storage. Biovance® Tri-layer provides a protective cover and supports the body's wound healing processes. Biovance® Tri-layer is currently being marketed as 3L BiovanceR and BiovanceR 3L Ocular.

Biovance® Tri-layer, Decellularized, Dehydrated Human Amniotic Membrane (DDHAM) consists of amniotic membrane that is stripped from the placenta and placed into a mild detergent, 1% Deoxycholic acid, for soaking. The amnion membrane undergoes mechanical scraping intended to remove nearly 100% of the amniocytes and chorionic cells from the surface of the membrane. The vast majority of the fibroblasts are also removed from the substance of the tissue. This membrane is layered onto itself and dried to create a three-layered product version of Biovance R. The final product is a structural tissue composed of extracellular matrix that retains the native collagen structure of amniotic membrane, including fibronectin that binds to collagen and other matrix components. The finished product is a tri-layer amniotic membrane, devoid of cells, hormones, growth factors, and cytokines.

To streamline and optimize the manufacturing process of BiovanceR Tri-layer, the process development team leveraged all the processing steps for the Biovance R process. The layering step was added before drying the membrane. Sterilization steps and release criteria were also carried over from the BiovanceR process to the BiovanceR Tri-layer process.

The amniotic membrane is harvested into a 1% Deoxycholic Acid solution and can be stored at 2-8o C for up to 14 days. Once acceptable maternal blood results have been received, the amnion is removed from storage and processing initiates. The Amnion undergoes a series of manual scrapings and washes before being layered. The amnion is layered so that it is three layers thick with the stromal side outward on the top and the bottom. Once dry the amnion is cut into the desired sizes, each individual piece is placed into an inner pouch, labeled, sealed, and submitted for visual inspection. During visual inspection, the tissue is inspected for size, shape, holes, rips/tears, debris, and stains. After visual inspection, each piece in the inner pouch is placed into a labeled outer pouch, sealed, and sterilized.

### Results:

### Cell Adhesion:

Cell adhesion was greater on the stromal side (12,342.42±4,536.60 AU) than on the epithelial side of AMs (9,788.50±5,704.17 AU) (side main effect, F(1,18)=6.714, p=0.018), which can be attributed to the lower cell adhesion on the epithelial side of DHAM (4,247.75±2,732.87 AU), compared with the stromal side of DHAM (13,100.25±4,675.24 AU, p=0.017), the epithelial side of DDHAM (16,725.25±1,453.62 AU, p<0.001), the epithelial side of CHAM (8,392.50±1,425.86 AU, p<0.001), the stromal side of DDHAM (16,334.75±591.85 AU, p=0.002), and the stromal side of CHAM (7,592.25±1,073.22 AU. p<0.001) (side×AM, p=0.001).

Additionally, there was a significant difference in cell adhesion between AMs (AM main effect, F(2,18)=30.896, p<0.001), with significantly greater cell adhesion on DDHAM (16,530.00±1,048.46 AU) than on DHAM (8,674.00±5,912.61 AU, p<0.001) and CHAM (7,992.38±1,244.16 AU, p<0.001). However, as indicated above, cell adhesion varied with side and AM. Cell adhesion was similar between the epithelial side of DDHAM and the stromal side of DDHAM (p=0.645) and between the epithelial side of CHAM and the stromal side of CHAM (p=0.404). However, cell adhesion was significantly greater on the stromal side of DHAM than the epithelial side of DHAM (P=0.017). Therefore, cell adhesion was significantly greater on the stromal and epithelial side of DDHAM than the epithelial side of DHAM (p≤0.002), the epithelial side of CHAM (post hoc tests, p<0.001), and the stromal side of CHAM (post hoc tests, p<0.001), while cell adhesion was similar between the stromal side of DDHAM and the stromal side of DHAM (p=0.219).

TABLE 4
Cell adhesion by side and amniotic membrane.
Means and standard deviations are provided.

| Amniotic | | | |
|---|---|---|---|
| Membrane | Epithelial Side | Stromal Side | Total |
| DDHAM | 16,725.25 ± | 16,334.75 ± | 16,530.00 ± |
| | 1,453.62 | 591.85 | 1,048.46 |
| DHAM | 4,247.75 ± | 13,100.25 ± | 8,674.00 ± |
| | 2,732.87 | 4,675.24* | 5,912.61 |
| CHAM | 8,392.50 ± | 7,592.25 ± | 7,992.38 ± |
| | 1,425.86 | 1,073.22 | 1,244.16 |
| TOTAL | 9,788.50 ± | 12,342.42 ± | 11,065.46 ± |
| | 5,704.17 | 4,536.60* | 5,206.33 |

*Statistically significant difference between epithelial side and stromal side.

### Cell Proliferation:

Although the number of viable cells significantly declined over 7-day culturing (time main effect: (F(2,54)=44.880, p<0.001), cell number significantly varied with side, AM, and time (side×AM×time interaction; (F(4,54)=3.633, p=0.011). Most notably, cell number declined for all variables across time, except for the stromal side of DDHAM on day 4. On day 4, the relative proliferation rate was significantly greater on the stromal side of DDHAM (115.29±15.54%) than on the epithelial side of DDHAM (52.27±14.41%, p<0.001), the epithelial side of DHAM (12.54±16.79%, p=0.012), and the stromal side of CHAM (15.00±6.73%, p<0.001). There was no significant difference in the relative proliferation rate between the stromal side of DDHAM and the epithelial side of CHAM (46.83±25.69%, p=0.731) or between the stromal side of DDHAM and the stromal side of DHAM (95.54±44.25%, p=0.430). However, the stromal side of DHAM was significantly greater than the stromal side of CHAM (p=0.012). Despite a decline in cell number from day 4, on day 7, the relative proliferation rate was significantly greater on the stromal side of DDHAM (59.47±28.48%) than on the stromal side of CHAM (6.87±1.77%, p=0.035) and the epithelial side of DHAM (7.54±5.84%, p=0.017).

The number of cells was also significantly greater on the stromal side (9,383.33±6,469.15 AU) than on the epithelial side of AMs (5,648.00±5,312.56 AU, main effect side; F(1,54)=39.545, p<0.001), which is largely driven by significantly more cells on the stromal side than the epithelial side of DDHAM and DHAM (side×AM interaction: p<0.001); DDHAM Stromal: 14,972.00±4,973.00 AU vs DDHAM Epithelial: 10,438.50±5,555.98 AU, p=0.047; DHAM Stromal: 10,103.33±4,336.49 AU vs DHAM Epithelial: 1,590.42±2,431.25 AU, t(22)=5.932, p<0.001). Conversely, CHAM a similar number of cells on the epithelial side (4,915.08±3,072.42 AU) and the stromal side (3,074.67±3,401.09 AU, p=0.178).

Cell number was also significantly different between AMs (main effect AM; F(2,54)=79.570, p<0.001) with significantly more cells on DDHAM (12,705.25±5,652.67 AU) than on CHAM (3,994.88±3,306.13 AU, p<0.001). There was no significant difference in cell number between DDHAM and DHAM (5,846.88±5,543.10, P=0.065) or between DHAM and CHAM (p=0.085). The similar cell count for DHAM and CHAM can be explained by the low cell count on the epithelial side of DHAM (1,590.42±2,431.25 AU), which was significantly lower than the stromal side of DHAM (10,103.33±4,336.49 AU, p<0.001), the stromal side of DDHAM (14,972.00±4,973.00 AU, p<0.001), the epithelial side of DDHAM (10,438.50±5,555.98 AU, p<0.001), and the epithelial side of CHAM (4,915.08±3,072.42 AU, p=0.008). The cell count on the epithelial side of DHAM and the stromal side of CHAM were similar (3,074.67±3,401.09 AU, p=0.117).

TABLE 5
Cell proliferation by side, amniotic membrane,
and time. Means and standard deviations are provided.
Cell proliferation measured in fluorescent intensity (AU).

| Side & | | | | |
|---|---|---|---|---|
| Amniotic | | | | |
| Membrane | DAY 1 | DAY 4 | DAY 7 | TOTAL |
| Epithelial | | | | |
| Side | | | | |
| DDHAM | 16,725.25 ± | 8,679.25 ± | 5,911.00 ± | 10,438.50 ± |
| | 1,453.62 | 2,092.53 | 4,747.52 | 5,555.98 |
| DHAM | 4,247.75 ± | 279.50 ± | 244.00 ± | 1,590.42 ± |
| | 2,732.87 | 205.39 | 197.88 | 2,431.25 |
| CHAM | 8,392.50 ± | 3,884.50 ± | 2,468.25 ± | 4,915.08 ± |
| | 1,425.86 | 2,025.36 | 1,719.06 | 3,072.42 |
| Epithelial | 9,788.50 ± | 4,281.08 ± | 2,874.42 ± | 5,648 ± |
| Side Total | 5,704.17 | 3,903.66 | 3,590.64 | 5,312.56 |
| Stromal | | | | |
| Side | | | | |
| DDHAM | 16,334.75 ± | 18,852.25 ± | 9,729.00 ± | 14,972.00 ± |
| | 591.85 | 2,882.54 | 4,776.66 | 4,973.00 |
| DHAM | 13,100.25 ± | 10,992 ± | 6,217.75 ± | 10,103.33 ± |
| | 4,675.24 | 1,830.40 | 3,253.52 | 4,336.49 |
| CHAM | 7,592.25 ± | 1,102.00 ± | 529.75 ± | 3,074.67 ± |
| | 1,073.23 | 442.57 | 175.43 | 3,401.09 |
| Stromal | 12,342.42 ± | 10,315.42 ± | 5,492.17 ± | 9,383.33 ± |
| Side Total | 4,536.60 | 7,795.43 | 4,979.13 | 6,469.15 |
| TOTAL | 11,574.50 ± | 7,298.25 ± | 4,183.29 ± | 7,515.67 ± |
| | 5,206.34 | 6,771.29 | 4,450.91 | 6,170.94 |

### Cell Migration:

Cell migration significantly differed between AMs (AM main effect; F(2,49)=6.819, p=0.002), with cell migration significantly greater on DDHAM (466,085.13±98,339.52 px²) than CHAM (344,471.06±106,094.18 px², p=0.003). In addition, cell migration was significantly lower on the medium control than DDHAM (p<0.001), DHAM (420,349.88±95,109.86 px², p<0.001), and CHAM (p<0.001). There was no main effect of side, which indicates cell migration was similar on the epithelial (421,669.96±113,435.95 px²) and stromal sides of the AMs (389,934.08±107,979.51 px², F(1,49)=0.701, P=0.407). Cell migration was not statistically different across amniotic membrane and side (p=0.159).

TABLE 6
Migration Area. Counts, means, and standard deviations
are provided. Migration area is reported as px².

| Amniotic | | |
|---|---|---|
| Membrane & Side | Cell Migration | |
| DDHAM | | |
| Epithelial Side | 482,961.50 ± | 99,654.98 |
| Stromal Side | 449,208.75 ± | 100,701.20 |
| DDHAM Total | 466,085.13 ± | 98,339.52 [symbol] |
| DHAM | | |
| Epithelial Side | 461,119.13 ± | 90,282.12 |
| Stromal Side | 379,580.63 ± | 86,220.78 |
| DHAM Total | 420,349.88 ± | 95,109.86 [symbol] |
| CHAM | | |
| Epithelial Side | 320,929.25 ± | 80,791.60 |
| Stromal Side | 368,012.88 ± | 127,772.77 |
| CHAM Total | 344,471.06 ± | 106,094.18* [symbol] |
| Medium Control | 145,349.00 ± | 58,822.77 |
| TOTAL | 372,451.59 ± | 139,865.11 |

*Statistically significant difference compared with DDHAM. [symbol]
Statistically significant difference compared with medium control.

### Example 4

### A Decellularized Dehydrated Human Amniotic Membrane-Derived Biomaterial Supports Human Corneal Epithelial Cell Function and Inflammatory Response

Statement of Purpose: Successful application of decellularized tissue-based biomaterials for wound healing requires matrix components that support cell function and differentiation. Amniotic membrane (AM) is a naturally derived biomaterial from human placental tissue with unique biological and mechanical properties that render it suitable for use in ocular healing (1,2). The purpose of this study is to evaluate the effects of sidedness and AM processing methodology on human corneal epithelial cell (HCEC) function in vitro. Experimental variables include AM sidedness (epithelial [E] and stromal [S]) and AM processing methodology (decellularized and dehydrated [DDHAM], dehydrated [DHAM], and cryopreserved [CHAM]). Dependent variables include HCEC viability, migration, and inflammatory response.

Methods: Three differently processed, commercially available ocular AMs were selected: Biovance3L Ocular (DDHAM), Ambio2® (DHAM), and AmnioGraft® (CHAM). HCECs were seeded onto the E and S sides of AMs and incubated for 1, 4 and 7 days. Cell viability was measured at each time point on the AMs using alamarBlue assay. Conditioned media from HCECs cultured on the AMs were collected, and the effect of conditioned media on the migration of HCECs was evaluated using a scratch wound assay. An inflammatory response was induced by TNFa treatment. The effect of AM on the expression of pro-inflammatory genes in HCECs was compared using quantitative polymerase chain reaction (qPCR). The significance level for all statistical tests was set at p=0.05. Cell viability was analyzed with a two-way analysis of variance (ANOVA), cell proliferation with a three-way ANOVA, and mRNA expression with a one-way ANOVA. Tukey's and unpaired t-tests were used for post-hoc analyses.

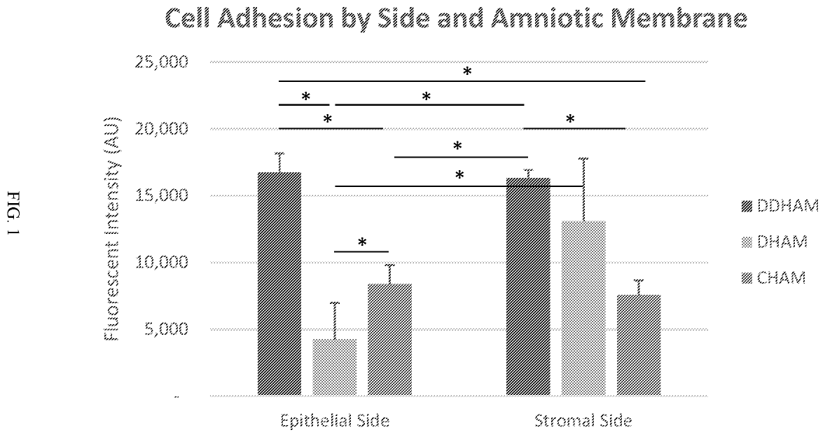

Results: On day 1, cell viability was significantly higher on DDHAM-E&S than CHAM-E&S (p<0.001) and DHAM-E (p≤0.002). On day 4, cell viability was significantly higher on DDHAM-S than all other variables (p≤0.004, FIG. 1). In addition, on day 4, cell viability was comparable

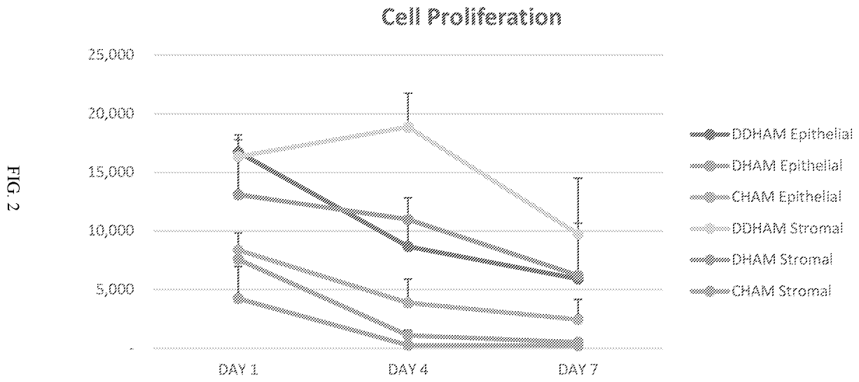

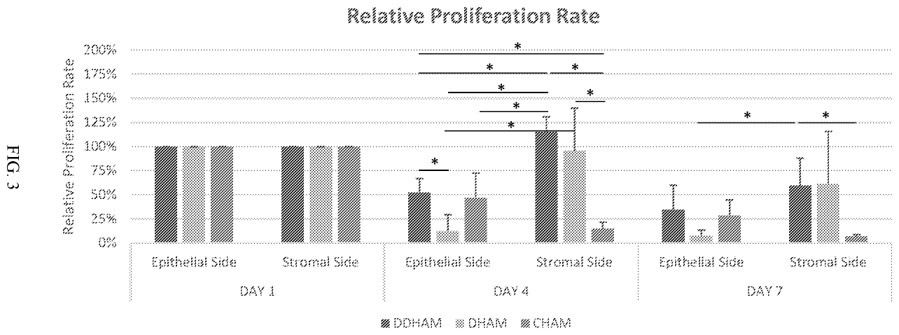

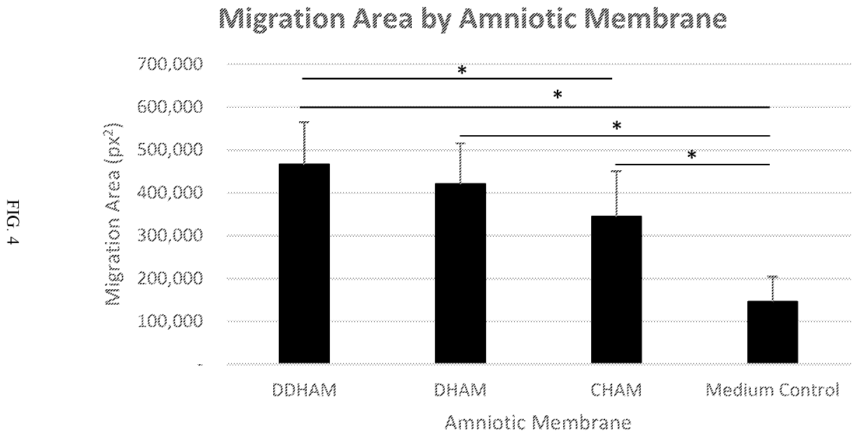

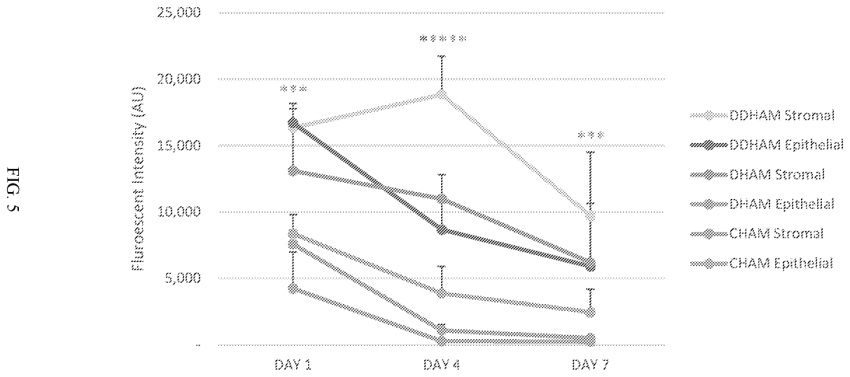

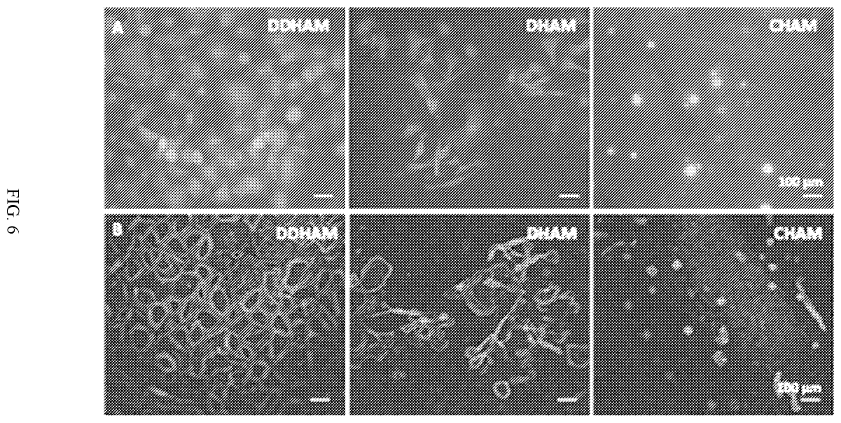

between DDHAM-E and DHAM-S (p=0.147) and significantly higher than DHAM-E (p≤0.004), CHAM-S&E (p≤0.017). On day 7, cell viability was significantly higher on DDHAM-S than DHAM-E (p=0.028) and CHAM-S&E (p≤0.049). Cell viability was similar between DDHAM-E and all other variables (p≥0.097). HCEC migration in the presence of conditioned media from cells cultured on DDHAM and DHAM was comparable (p=0.885) and significantly greater than cells grown on CHAM (p≤ 0.005). Interestingly, HCECs cultured on DDHAM adapted a cobblestone morphology (FIG. 2), which mimics the morphology of ocular epithelial cells in situ (3). The migration of HCEC in the presence of conditioned media from cells cultured on ocular scaffolds was significantly greater than control conditioned media from cells grown on tissue culture plastic (p<0.001). Moreover, in response to inflammatory stimulation by TNFa, the gene expression of pro-inflammatory cytokines (IL-6, IL-8, and TNFa) in HCECs on DDHAM showed an initial increase followed by a decline across time (FIG. 3).

Conclusion: In this in vitro study, DDHAM-S best supported HCEC viability and migration. The presence of DDHAM also attenuated the inflammatory response of HCECs over time.

### References

1. Walkden A. Clin Ophthalmol. 2020: 14:2057-2072.
2. Malhotra C. World J Transplant. 2014:4(2): 111-121.
3. Sosnová-Netuková M. Br J Ophthalmol. 2007:91(3):372-378.

### Example 5

### An In-Vitro Comparison of Human Corneal Epithelial Cell Activity and Inflammatory Response on Differently Designed Ocular Amniotic Membranes and Clinical Case Study

Amniotic membrane (AM) is a naturally derived biomaterial with biological and mechanical properties important to Ophthalmology. The epithelial side of the AM promotes epithelialization, while the stromal side regulates inflammation. However, not all AMs are equal. AMs undergo different processing with resultant changes in cellular content and structure. This study evaluates the effects of sidedness and processing on human corneal epithelial cell (HCEC) activity and the effect of processing on HCEC inflammatory response and then presents a case study. Three differently processed, commercially available ocular AMs were selected: (1) Biovance3L Ocular, a decellularized, dehydrated human AM (DDHAM), (2) AMBIO2®, a dehydrated human AM (DHAM), and (3) AmnioGraft®, a cryopreserved human AM (CHAM). HCECs were seeded onto the AMs and incubated for 1, 4 and 7 days. Cell adhesion and viability were evaluated using alamarBlue assay. HCEC migration was evaluated using a scratch wound assay. An inflammatory response was induced by TNFa treatment. The effect of AM on the expression of pro-inflammatory genes in HCECs was compared using quantitative polymerase chain reaction (qPCR). Staining confirmed complete decellularization and the absence of nuclei in DDHAM. HCEC activity was best supported on the stromal side of DDHAM. Under inflammatory stimulation, DDHAM promoted a higher initial inflammatory response with a declining trend across time. Clinically, DDHAM was used to successfully treat anterior basement membrane dystrophy. Compared with DHAM and CHAM, DDHAM had significant positive effects on the cellular activities of HCECs in vitro, which may suggest greater ocular cell compatibility in vivo.

Introduction: Amniotic membrane (AM) is a naturally derived biomaterial with unique biological and mechanical properties that render it particularly suitable for use in ophthalmology (Leal-Marin et al. 2021; Walden, 2020; Liu et al. 2019; Malhotra & Jain, 2014; Fernandes et al. 2005). Amnion tissue is thought to promote healing and reconstruction of the ocular surface through the promotion of epithelialization (Shayan et al. 2019; Meller et al. 2002: Meller et al. 1999), reduction of inflammation (Sharma et al. 2016; Tabatabaei et al. 2017: Tandon et al. 2011), inhibition of scar tissue formation (Niknejad et al. 2008, Tseng et al. 1999 Lee et al. 2000), blockage of new blood vessels (Hao et al. 2000), and the ability to act as an antimicrobial agent (Mamede & Botelho, 2015; Tehrani et al. 2013; Sangwan et al. 2011: Kjaergaard et al. 2001: Kjaergaard et al. 1999, Inge et al. 1991). In ophthalmology, the AM is widely used to treat a variety of ocular conditions. Clinically, the AM can be used as a surgical patch, as a substrate to replace damaged ocular tissue, or in combination as both a patch and a substrate.

As a patch, the AM acts as a temporary biological bandage or contact lens, promoting re-epithelization of the host tissue beneath the patch (Walden, 2020, Malhotra & Jain, 2014) and is placed stromal side down to downregulate the inflammatory response by trapping inflammatory cells and inducing apoptosis (Dua et al. 2004; Shimmura et al. 2001). By placing the AM epithelial side up, the AM acts as a substrate and scaffold for epithelial cell migration and growth (Malhotra & Jain, 2014). Although it is widely accepted that the AM should be placed epithelial side up to promote re-epithelialization (Hu et al. 2003), the stromal side of the membrane has been shown to support epithelial cell growth (Seitz et al. 2006). Notably, much of the existing research is limited to cryopreserved AMs, and it remains unclear whether these findings also apply to other AMs that have undergone different processing methodologies.

Prior to clinical application, the AM is sterilized and processed with resultant changes to cellular content and structure (Leal-Marin et al. 2021: von Versen-Höynck et al. 2004; Lim et al. 2010). This tissue can be used directly, or it can undergo the additional process of decellularization (Tehrani et al. 2021). Decellularization is a process whereby endogenous cells, cell debris, and DNA remnants are removed to prevent an immune response, while retaining the natural structural and chemical elements of the extracellular matrix (ECM) (Gholipourmalekabadi et al. 2015). Previous studies have demonstrated a correlation between the quantity of residual DNA in ECM products and the host inflammatory response (Keane et al. 2012; Seif-Naraghi et al. 2013). As with the preservation of tissue, decellularization can also affect the structures and entities within the ECM (Aamodt & Grainger, 2016). Therefore, successful preservation-decellularization protocols must delicately balance the removal of cellular material and the retention of the innate properties and functional characteristics of ECM (Gholipourmalekabadi et al. 2015; Aamodt & Grainger, 2016; Balestrini et al. 2015). To our knowledge, no studies have evaluated how differing preservation-decellularization protocols affect the cellular activity and inflammatory response of human corneal epithelial cells (HCECs).

For the first time, this project aims to evaluate:
the effect of amniotic membrane sidedness (i.e., epithelial vs stromal) and processing methodology on the cellular activities of HCEC (i.e., adhesion, viability, and migration), the effect of different processing methodologies on the inflammatory response of HCECs (i.e., expression of pro-inflammatory cytokines).

Therefore, three differently processed, commercially available ocular AMs were used for comparison:
Biovance3L Ocular (Celularity, Florham Park, N.J.), a decellularized, dehydrated human amniotic membrane (DDHAM),
AMBIO2® (Katena, Parsippany, N.J.), a dehydrated human amniotic membrane (DHAM), AmnioGraft® (Biotissue, Miami, Fl.), a cryopreserved human amniotic membrane (CHAM).

Biovance®3L Ocular is a three-layer DDHAM. It is designed uniquely with the stromal side facing out. Therefore, the stromal side interfaces with the ocular surface regardless of its orientation. Furthermore, having three layers enhances its handling properties. The AM is excised from qualified term placentas, washed, and scraped to remove extraneous tissues and cells. The tissue is then decellularized using an osmotic shock followed by a mild detergent treatment, dried, and sterilized. Previous research has confirmed that this proprietary decellularization process removes residual cells, cell debris, growth factors, and cytokines, while retaining an ECM structure with high collagen content and key bioactive molecules, such as fibronectin, laminin, glycosaminoglycans, and elastin (Bhatia et al. 2007).

AMBIO2® is a single-layer, aseptically processed DHAM. The dehydration process removes moisture, while preserving the structural matrix and biological components of the tissue (Instructions for Use, 2021), including growth factors and cytokines.

AmnioGraft® is a single-layer CHAM. The AM is preserved using a proprietary cryopreservation method, CRYOTEK®. The cryopreservation preservation process renders the amniotic epithelial cells nonviable, while maintaining an intact cellular structure and preserving growth factors and cytokines (Rodriguez-Ares et al. 2009).

DDHAM retains its native ECM and is devoid of all cellular components, DNA, growth factors and cytokines. Therefore, the authors hypothesize that DDHAM will provide a more cell-friendly matrix supporting the cellular activity and inflammatory response of HCECs compared with the two other ocular AMs containing residual DNA and other cellular components. Results from this in vitro study will further the basic understanding of how the preservation and decellularization of amnion tissue affects the activity of human ocular epithelial cells. It also has the potential to elucidate the clinical application of DDHAM to support corneal and conjunctival related injuries or defects, such as corneal epithelial defect healing, pterygium repair, fornix reconstruction, and other ocular procedures.

Materials & Methods: Since the testing materials are commercially available products and this study did not require direct interaction with human subjects (donors), institutional review board approval was not required.

Ocular AMs: Three ocular AMs were used in this study: DDHAM, DHAM, and CHAM. DDHAM (Lot # OCLR0010) and DHAM samples were stored at room temperature. CHAM samples were stored at −80° C. All AMs were handled according to the manufacturer's instructions. DDHAM samples came as individually packaged 10 mm discs. Therefore, 10 mm discs were made from DHAM sheet, using a 10 mm biopsy punch (Thermo Fisher Scientific, Waltham, Mass., USA). Each piece (5 cm×10 cm) of CHAM was thawed and washed in 20 mL of phosphate buffered saline (PBS) in a petri dish for 10 minutes (min) to remove the cryoprotectants and 10 mm discs were made from the washed AMs using 10 mm biopsy punch. DDHAM is multilayered (three layered) with stromal side of AM facing out on both sides. To evaluate the sidedness of DDHAM, a differently designed version was prepared (three layered) with epithelial side of AM facing out on both sides, DDHAM(E). 10 mm discs of each AM sample were placed in the wells of a 48-well plate (1 disc/well) (Cell-Repellent 48-Well Microplate, Greiner Bio-One, Monroe, N.C., USA) with either stromal side or epithelial side of AM in contact with cells. A sterile O-ring (McMaster-Carr, Robbinsville, N.J., USA), measuring 2 mm in width with 7 mm inner diameter, was placed on the top of each AM to hold the AM in place. Amniotic membranes were pre-conditioned with growth medium (0.4 mL/well) at 37° C. for 2 hours (h) before they were seeded with cells. At least two lots (donors) of each type of AM were used in this study. In each independent experiment, four samples (n=4) from each AM were used, of which two samples were from one lot and two samples were from another lot. At least two independent experiments were performed for each individual assay.

Primary cells: The human corneal epithelial cells (HCECs, Cat#PCS-700-010 Lot# 80915170), corneal epithelial cell base medium, and corneal epithelial cell growth kit were purchased from ATCC (Manassas, Va., USA). The complete growth medium for HCECs was prepared according to the manufacturer's instructions.

Assessment of cell adhesion to AMs: HCECs at passage 4 (P4) were cultured to 80% confluence in 10 cm cell culture dishes following the manufacturer's instructions. Cells were rinsed once with 5 mL phosphate-buffered saline (PBS)/dish. One milliliter of 0.25% trypsin (Thermo Fisher Scientific, Waltham, Mass., USA) was added to each dish and incubated at 37° C. for 5 min. Two milliliters of minimum essential medium-alpha (Thermo Fisher Scientific, Waltham, Mass., USA) medium containing 10% FBS was added to the dish to neutralize the trypsin. Cells were transferred to 15 mL conical tubes and centrifuged at 1000 RPM (Revolutions Per Minute) for 5 min. Cells were re-suspended in complete growth medium and counted using a hemocytometer.

HCECs (2×104/well) were added to each well containing pre-conditioned AMs. The plates were incubated at 37° C. with 5% CO2 and 95% humidity. After incubation for 24 h, the media were removed, and the cells were washed once with PBS. The viability of adhered cells was detected using the alamarBlue assay. Briefly, 0.2 mL/well of alamarBlue solution, consisting of complete growth medium+10% alamarBlue reagent (Bio-Rad, Hercules, Calif., USA) was added to each well and incubated at 37° C. for 45 min After incubation, 0.1 mL/well of supernatant was transferred to a 96-well plate. Fluorescence intensity was measured using a multimode microplate reader (SparkR), TECAN, Switzerland) at excitation/emission (Ex/Em)=540 nm/590 nm. The fluorescence intensity was expressed in arbitrary units (AU).

Staining of AMs and cells: To visualize the structural features of AMs, three different AMs were rehydrated, washed, and embedded in Tissue-Tek O.C.T. compound (Sakura, Torrance, Calif., USA) vertically. Five micron/slice cryosections were made using Leica CM1850 cryostat (Leica Biosystems, Buffalo Grove, Ill., USA). The cryosections on microscope slides were fixed with 4% paraformaldehyde for 1 h and permeabilized in 0.5% Triton X100 in PBS for 1 h. The fixed and permeabilized samples were stained with anti-human type I antibodies (ab34710, Abcam, Cambridge, Mass., USA) overnight. Samples were then stained with Alexa Fluor 555-anti-rabbit IgG, Alexa 488-Phalloidin (Life Technology, Carlsbad, Calif., USA) and Hoechst dye 33258 (Thermo Fisher Scientific, Waltham, Mass., USA) for 60 min. After staining, a coverslip was mounted onto each sample in the presence of ProLong Gold Antifade Mountant (Thermo Fisher Scientific, Waltham, Mass., USA).

To visualize the viable cells on different AMs, HCECs were cultured on different AMs as described in “Assessment of Cell Adhesion to Amniotic Membranes” for 1 or 4 days. At each time point, the medium was removed from each well, and 0.2 mL/well of fresh complete growth medium containing 50 nM Calcein AM (Thermo Fisher Scientific, Waltham, Mass., USA) was added to each well. After incubation for 30 min at 37° C., the medium was removed. Cells were washed twice with PBS and ready to be imaged.

To visualize the cell morphology, HCEC cells cultured on different AMs for 4 days were fixed with 4% paraformaldehyde for 1 h and permeabilized in 0.5% Triton X100 in PBS for 1 h. The fixed and permeabilized cells were stained with Alexa 488-Phalloidin (Life Technology, Carlsbad, Calif., USA) for 30 min and observed under an epi-fluorescent microscope (Zeiss Observer D1, Jena, Germany).

H&E staining of AMs: Cryosections of AMs were baked at 60° C. overnight, fixed in 4% paraformaldehyde for 30 min, and rinsed three times with PBS. Samples were stained in Harris Hematoxylin Solution (Sigma-Aldrich, Inc., St. Louis, Mo.) for 10 min and rinsed in running tap water for 1 min. Slides were then immersed two times in differentiation solution (0.25 mL concentrated Hydrochloric Acid to 100 mL of 70% alcohol). Subsequently, slides were rinsed under running tap water for 1 min, followed by immersion in Scott's Tap Water Substitute (1% Magnesium sulfate (MgSO4) and 0.06% Sodium Bicarbonate) for 60 seconds. After a 30 second wash in 95% reagent alcohol, samples were counterstained in Alcoholic Eosin Y Solution (Sigma-Aldrich, Inc., St. Louis, MO 68178) for 10 min. Upon completion of staining, slides were dehydrated by three washes in 100% absolute ethanol, followed by three Histoclear II washes. Slides were mounted using Permount mounting medium (Fisher Scientific Inc.) and imaged using Zeiss Axio Observer Al microscope.

Assessment of cell viability on AMs over time: HCECs (1×104/well) were added to each well of 48-well plates containing pre-conditioned AMs. Three sets of plates for each cell type were set up and incubated at 37° C. with 5% CO2 and 95% humidity for 1, 4, and 7 days. At the first time point, the medium from each well of all plates was removed, and fresh medium was added. The viability of cells in the first set of plates was measured using the alamarBlue assay. The second and third sets of plates were cultured at 37° C. At the second time point, the viability of cells in the second set of plates was measured. The third set of plates was cultured in fresh medium at 37° C. The viability of cells in the third set of plates was measured using the alamarBlue assay at the third time point.

Conditioned media for migration assay: In the test condition, HCECs (2×104/well) were added to each well of 48-well plates containing pre-conditioned AMs. In the control condition, no HCECs were added to the pre-conditioned AMs. After culturing for 24 h, the medium was removed. 0.4 mL/well of fresh growth medium was added to each well with or without cells and incubated at 37° C. for 24 h. The supernatants (24-h conditioned media) were collected from each well and immediately used for the migration assay. The stromal sides of AMs were used for this experiment.

Scratch wound migration assay: 5×104/well HCECs were added to each well of tissue culture-treated polystyrene 48-well plates and cultured at 37° C. with 5% CO2 and 95% humidity for 2 days. Scratch wounds were made on a confluent monolayer using the tip of a sterile metal rod. The medium was removed, and conditioned medium collected from cells cultured on AMs was added to the wound. Images of the wound areas were captured at 0 h. At minimum, four areas were monitored for each testing group. The plates were incubated at 37° C. for 24 h. The exact same wound areas (with marker reference) were imaged at 24 h. Wound areas were measured using ImageJ software (NIH) in arbitrary units (square pixels, px2). Migrated area=Area0 h-Area24 h.

Stimulation of inflammatory responses of HCECs: 2×104/well HCECs were seeded and cultured on different AMs for 24 h. Media were removed and fresh medium “−Tumor Necrosis Factor-alpha (TNF-□)” or fresh medium containing 10 ng/ml of human TNF-□ Cat#300-01A, PeproTech Cranbury, N.J.) “+TNF-□” were added to cells and incubated for 24 h, 48 h or 72 hr. At each time point, the supernatants were collected for multiplex analysis and the cells were lysed in 0.2 mL of RNA lysis buffer (Promega, Durham, N.C.) for quantitative polymerase chain reaction (qPCR) analysis as described below.

Assessment of relative mRNA expression by qPCR: The quantification of the relative gene expression of cytokines by qPCR was performed as previously described (Mao et al. 2021). Briefly, total RNA from cell lysates was purified using SV 96 Total RNA Isolation System (Promega). RNA concentration and purity were measured using TECAN Spark Nano plate (TECAN, Morrisville, N.C.). cDNA preparation and qPCR were performed as described (Mao et al. 2017). The primers for qPCR used for this study were from QuantiTect (Qiagen, Germantown, Md.): granulocyte-macrophage colony-stimulating factor (GM-CSF: QT00000896), interleukin 6 (IL-6: QT00083720), interleukin-8 (IL-8: QT00000322), Tumor Necrosis Factor alpha (TNF-□: QT01079561), and glyceraldehyde 3-phosphate dehydrogenase (GAPDH: QT01192646). Each sample was run in duplicate. After the run was completed, a second derivative analysis was performed using the raw data to determine the mean Cp (Crossing point-PCR-cycle) for each sample. For each gene expression, expression of GAPDH served as an internal control. Relative mRNA expression was determined by Pfaffl analysis (EACp target/EACp reference) in which primer efficiency E=10\hat{ }(−1/slope) and ΔCp=mean Cp of sample−mean Cp of Control. The expression of cells on tissue culture polystyrene (TCP) or the expression of cells at 24 h was used as “Control” for analyses, which was defined in the specific analysis in “Results”.

Statistical Methods: In the evaluation of HCEC activity, the independent variables were AM (DDHAM, DHAM, CHAM), side (epithelial, stromal), and time (day 1, day 4, and day 7). The dependent variables were cell adhesion, cell viability, and migration. In the evaluation of HCEC inflammatory response by mRNA expression, the independent variables were amniotic membrane (DDHAM, DHAM, CHAM, Control [TCP]), condition (resting, stimulated), and time (24 h, 48 h, and 72 h). In the evaluation of HCEC inflammatory response by protein levels, the independent variables were amniotic membrane (DDHAM, DHAM, CHAM, Control [TCP]) and condition (resting, stimulated, AM only). The dependent variables were relative mRNA expression of cytokines (GM-CSF, IL-6, IL-8 and TNF-α) and protein levels of cytokines and chemokines (GM-CSF, IL-1β, IL-IRA, IL-6, IL-8, TGFβ2, and VEGF).

All analyses were conducted using IBM SPSS (Build 1.0.0.1444). The significance level for all statistical tests was set at p=0.05. The data were tested and found to be normally distributed. Cell adhesion and migration were analyzed with a two-way analysis of variance (ANOVA) with Tukey post-hoc tests. Cell proliferation was analyzed with a three-way ANOVA with Tukey post-hoc tests. Relative mRNA expression at 24 h was analyzed with a two-way ANOVA with Tukey post-hoc tests to evaluate each dependent variable in each of the testing conditions. Relative mRNA expression across time was analyzed with a one-way ANOVA with Tukey post-hoc tests to evaluate each dependent variable in each of the testing conditions. Significant interactions were evaluated with simple main effects analysis with Sidak correction for multiple comparisons. Data are reported as mean±standard deviation (SD) within the text and FIGS.

### Results:

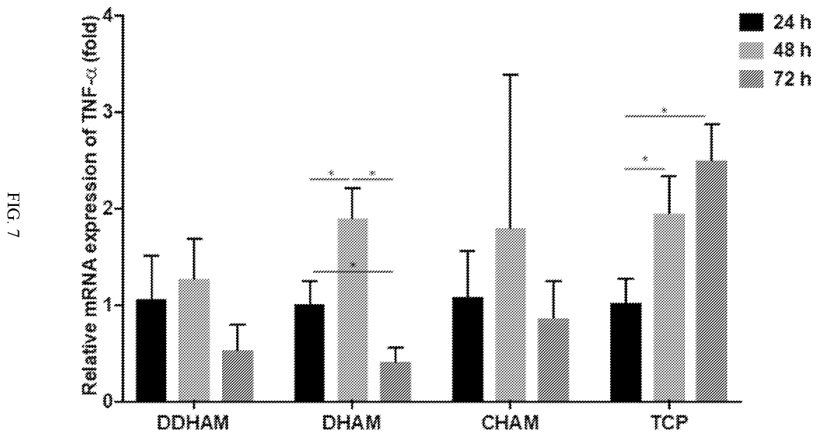

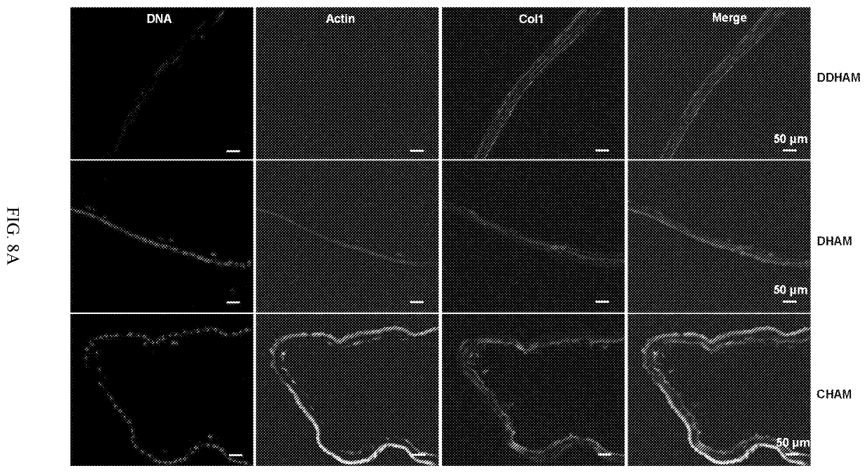

Structure of AMs: To evaluate the structures of these three AMs, cross-sections of AMs were stained for cellular components (DNA and actin) and ECM (type I collagen) (FIG. 8A). While strong nuclei staining and actin staining were detected in DHAM and CHAM, neither actin nor nuclei staining was detected in DDHAM. The presence of type I collagen was detected in all three AMs. H&E staining of the three AMs (FIG. 8B) confirmed complete decellularization and absence of nuclei in DDHAM, compared with DHAM and CHAM. DHAM showed meagre staining of dark blue nuclear remnants, while CHAM showed intact dark blue staining for nuclei, showing the presence of cells.

Adhesion of HCECs on different AMs: Cell adhesion on different AMs and different sides of AMs was evaluated by comparing the cell viability (reflecting the quantity of adhered cells) at 24 h. The fluorescence intensity was expressed in arbitrary units (AU).

Effect of sidedness. Cell adhesion was greater on the stromal side than on the epithelial side of AMs (side main effect, p=0.018), which can be explained by the lower cell adhesion on the epithelial side of DHAM, compared with the stromal side of DHAM (p<0.001: side×AM, p=0.001; FIG. 9). There was no significant difference between the epithelial and stromal sides of DDHAM (p=0.822) or between the epithelial and stromal sides of CHAM (p=0.645).

Effect of AM. Additionally, there was a significant difference in cell adhesion between AMs (AM main effect, p<0.001), with significantly greater cell adhesion on DDHAM than on DHAM (p<0.001) and CHAM (p<0.001). However, as previously indicated, cell adhesion varied with side and AM (p=0.001; FIG. 9). On the epithelial side, cell adhesion was significantly greater on DDHAM than on DHAM (p<0.001) and CHAM (p<0.001), and there was no significant difference between CHAM and DHAM (p=0.076). On the stromal side, cell adhesion was significantly lower on CHAM than on DDHAM (p<0.001) and DHAM (p=0.014), and there was no significant difference between DDHAM and DHAM (p=0.207). These results indicate that among these three AMs, the epithelial and stromal sides of DDHAM best supported cell adhesion.

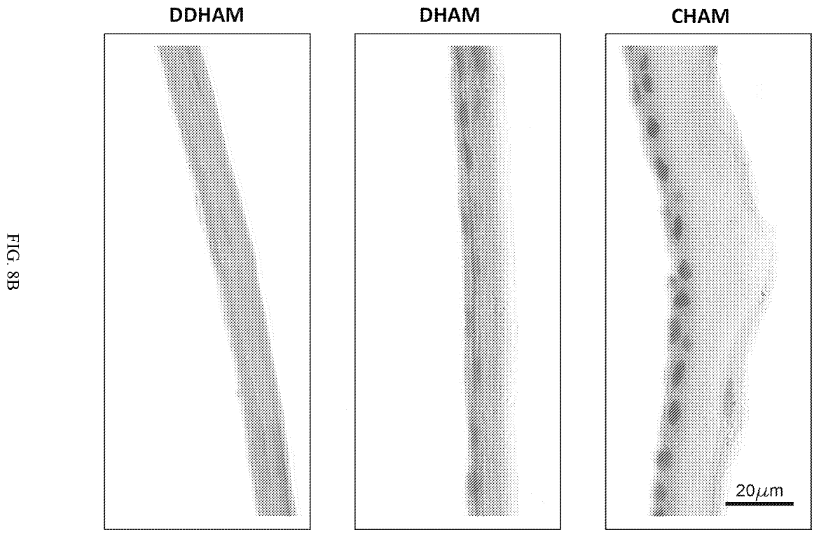

Viability and morphology of HCECs on different AMs on Day 4: Live Staining of Epithelial Cells. The viability of HCECs on the stromal side of different AMs (DDHAM, CHAM, DHAM) was observed 4 days after cell seeding (FIG. 10). Consistent with the quantitative results, the HCECs on DDHAM and DHAM appeared to have adhered and spread on day 4 after cell seeding, whereas HCECs on CHAM appeared to be disorganized and adopted a heterogeneous morphology. The morphology of HCECs on the AMs was monitored by actin staining on day 4 (FIG. 10). The HCECs on DDHAM adapted a cobblestone morphology with a dense actin ring structure.

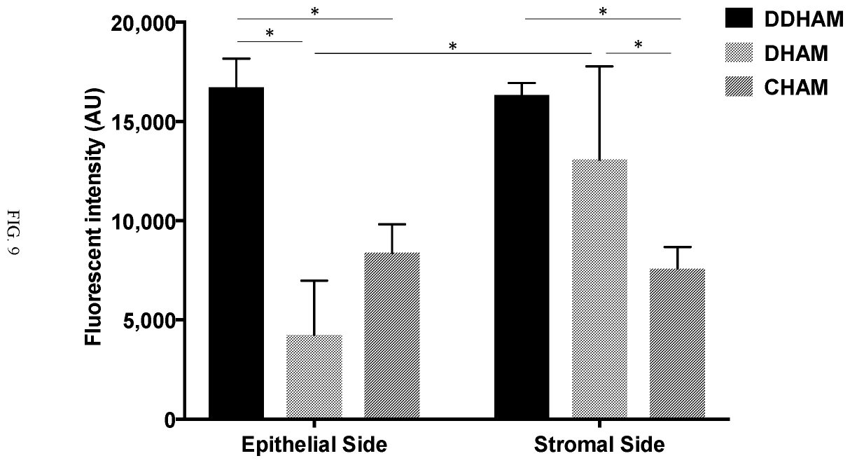

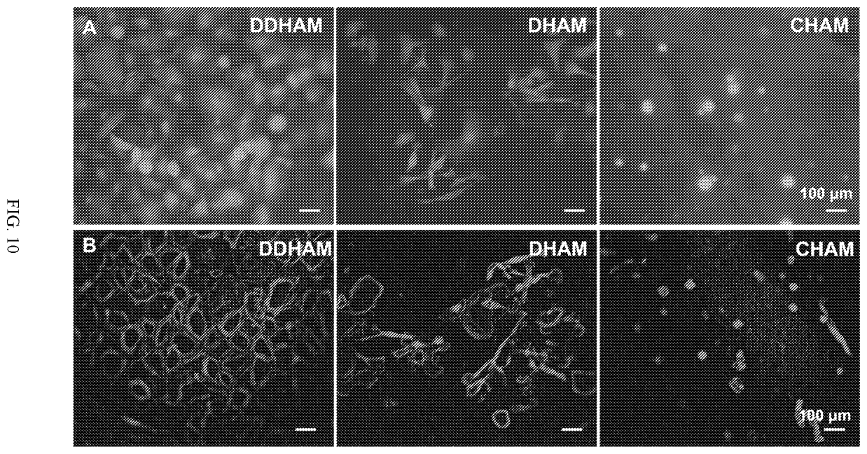

Cell viability on different AMs over time: The viability of cells on different AMs was monitored up to 7 days. Although the number of viable cells significantly declined over the 7-day culture (time main effect, p<0.001), cell viability significantly varied with side, AM, and time (side×AM×time interaction, p=0.011). Most notably, cell viability declined for all variables across time, except for the stromal side of DDHAM on day 4 (FIG. 11A).

Effect of sidedness. Cell viability was also significantly greater on the stromal side than on the epithelial side of AMs (main effect side, p<0.001), which can be explained by differences in relative cell viability between sides on days 4 and 7 (FIG. 11B). On day 4, the relative cell viability was significantly greater on the stromal side of DDHAM than the epithelial side of and DDHAM (p<0.001), and the relative cell viability was significantly greater on the stromal side of DHAM than the epithelial side of DHAM (p<0.001). Conversely, the relative cell viability was significantly greater on the epithelial side of CHAM than the stromal side of CHAM (p=0.039). On day 7, there were no significant differences in relative cell viability between the epithelial and stromal sides of DDHAM (p=0.102) or CHAM (p=0.157). However, the relative cell viability was significantly greater on the stromal side of DHAM than the epithelial side of DHAM (p<0.001).

Effect of AM. Cell number was also significantly different between AMs (main effect AM, p<0.001) with significantly more viable cells on DDHAM than on DHAM (p<0.001) and CHAM (p<0.001) and significantly more viable cells on DHAM than CHAM (p=0.036). The main effect of AM is largely explained by the significant differences in relative cell viability on days 4 and 7 (FIG. 11B).

On the epithelial side on day 4, the relative cell viability was significantly greater on DDHAM than on DHAM (p=0.032), meanwhile the relative cell viability was similar between DDHAM and CHAM (p=0.978) and between CHAM and DHAM (p=0.077). On the epithelial side on day 7, there were no significant differences between the three amniotic membranes (p≥ 0.219).

On the stromal side on day 4, the relative cell viability was significantly greater on DDHAM than on CHAM (p<0.001), and the relative cell viability was significantly greater on DHAM than on CHAM (p<0.001). There was no significant difference in the relative cell viability on the stromal side on day 4 between DDHAM and DHAM (p=0.477). On the stromal side on day 7, however, the relative cell viability was significantly lower on CHAM than DDHAM (p=0.003) and DHAM (p=0.002). As with the epithelial side, on the stromal side on day 7, there was no significant difference in the relative cell viability between DDHAM and DHAM (p=0.999).

The findings of higher cell viability on the stromal side of AMs and better maintenance of viability on DDHAM compared with DHAM and CHAM suggests that cell viability was best maintained on the stromal side of DDHAM.

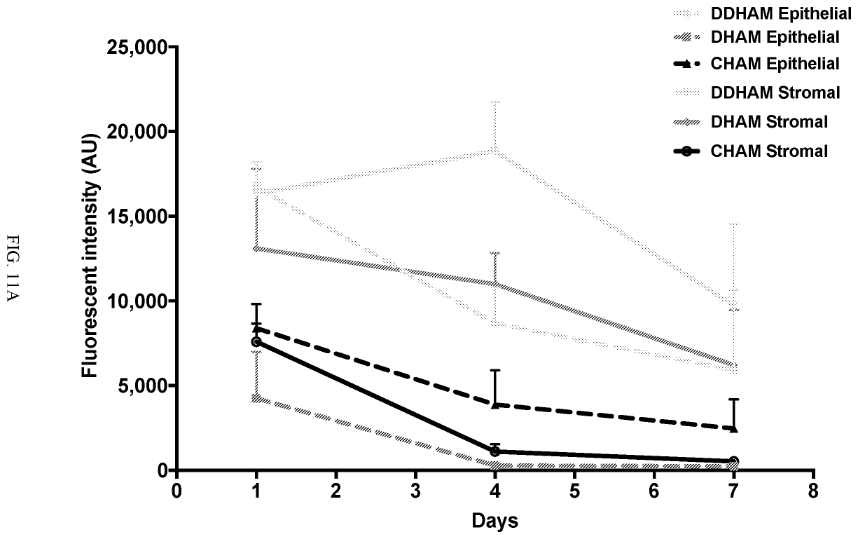

Migration of HCECs on different AMs: The conditioned media from different AMs in the absence of HCECs were tested to evaluate the effect of AM alone on the migration of HCECs. Additionally, differences in migration were compared between AMs in the presence of cells to determine if the factors released by HCECs cultured on different AMs affect cell migration. Cells cultured on AMs were conditioned for 24 h. The migration of HCECs in the presence of conditioned media from different AMs was evaluated using a scratch wound assay. Wound closure was monitored for 24 h (FIGS. 12A and 12B).

There was a significant interaction between the effects of amniotic membrane and the presence of cells (p=0.006; FIGS. 12A and 12B). Migration was significantly higher with cells than without cells on DDHAM (p=0.009) and DHAM (p<0.001). Migration was not significantly different with or without cells on CHAM (p=0.291) or on the control (p=0.265).

Effect of AM. Furthermore, among the conditioned media (CM) collected in the presence of cells, migration was significantly lower in CM from cells on CHAM than on DDHAM (p=0.004) and DHAM (p=0.002). There was no significant difference in migration between DDHAM and DHAM (p=1.000). Compared with the control in the presence of cells, migration was significantly higher in CM from cells on DDHAM (p<0.001), DHAM (p<0.001), and CHAM (p=0.005).

Gene expression of inflammatory cytokines in HCECs: Since the stromal side of AM has been reported to regulate the inflammatory response (Dua et al. 2004; Shimmura et al. 2001), the effect of the stromal side of these three AMs on the inflammatory responses of HCECs was evaluated. Cytokines with previously demonstrated roles in wound healing were selected, including GM-CSF, IL-6, IL-8 or TNF-□ (Rho et al. 2015; Arranz-Valsero et al. 2014; Ebihara et al. 2011; Nishida et al. 1992; Hafezi et al. 2018; Strieter et al. 1992; Koch et al. 1992: Wang et al. 2020; Yang et al. 2019). To this end, the inflammatory response of HCECs under an in vitro inflammatory condition was mimicked by the stimulation with TNF-□ for 24 h. The gene expression (relative mRNA levels) of GM-CSF, IL-6, IL-8, or TNF-□ in HCECs on different AMs was assessed by qPCR compared with the gene expression in cells cultured on standard cell culture surface, TCP.

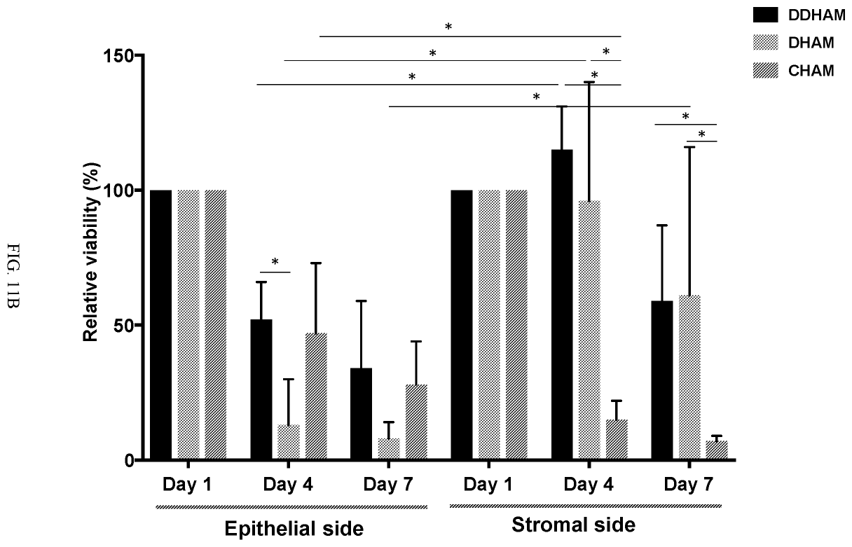

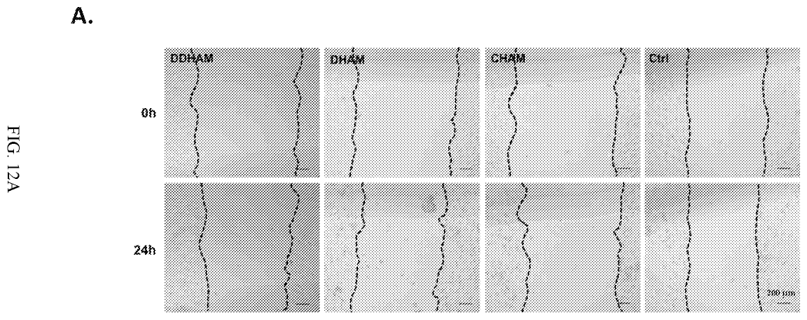

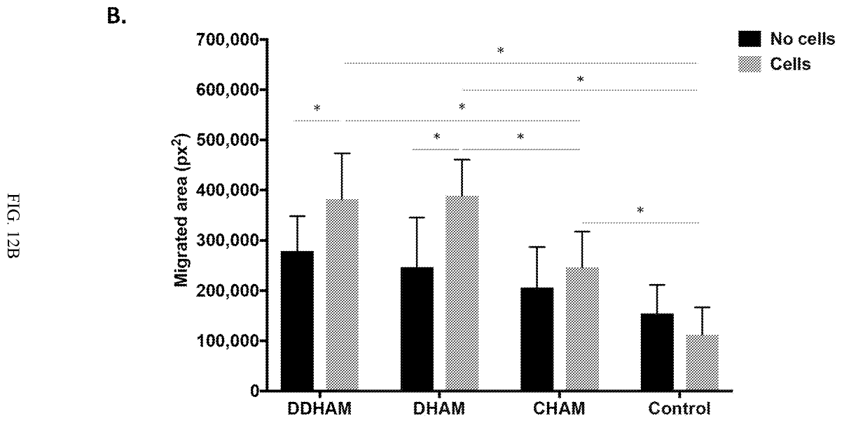

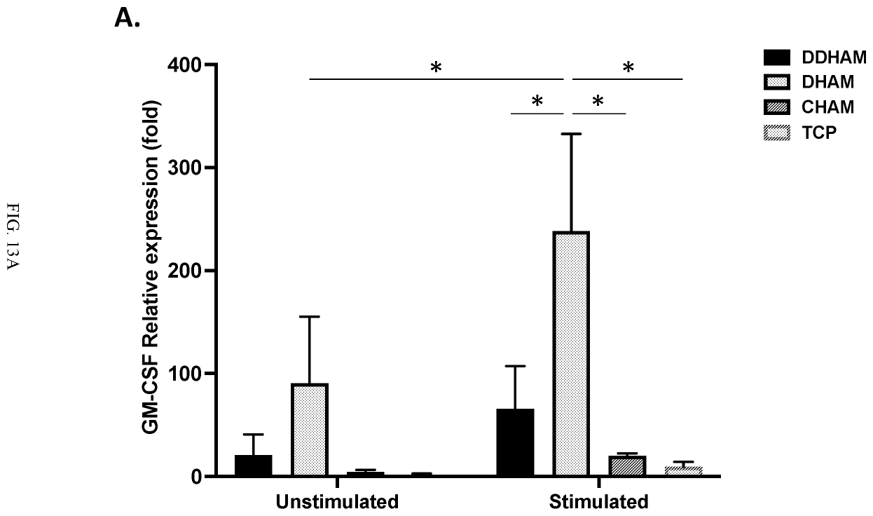

GM-CSF. The expression of GM-CSF at 24 h varied significantly by stimulation condition (±TNFα) and AM (p=0.049) (FIG. 13A). With stimulation, the expression of GM-CSF significantly increased on DHAM (p<0.001), but not DDHAM (p=0.226), CHAM (p=0.664), or TCP (p=0.827). Comparing the expression of GM-CSF between amniotic membranes in the resting condition showed a similar expression of GM-CSF on DDHAM, DHAM, CHAM, and TCP (p≥0.134). Comparing the expression of GM-CSF between amniotic membranes in the stimulated condition showed significantly greater expression on DHAM than on DDHAM (p=0.001), CHAM (p<0.001), and TCP (p<0.001).

IL-6. The expression of IL-6 at 24 h varied significantly by stimulation condition and amniotic membrane (p=0.002) (FIG. 13B). With stimulation, the expression of IL-6 significantly increased on DDHAM (p<0.001), CHAM (p=0.017), and TCP (p=0.014), but not DHAM (p=0.128). Comparing the expression of IL-6 between amniotic membranes in the resting condition showed a similar expression of IL-6 on DDHAM, DHAM, CHAM, and TCP (p≥0.717). In the stimulated condition, there was significantly higher expression of IL-6 on DDHAM than on DHAM (p<0.001), CHAM (p<0.001), and TCP (p<0.001). No other significant differences were found.

IL-8. Although the expression of IL-8 at 24 h did not vary significantly by stimulation condition and AM (p=0.188), there were main effects for stimulation condition (p<0.001) and AM (p=0.002). The overall expression of IL-8 significantly increased with stimulation. Post-hoc analyses revealed that overall IL-8 expression was significantly greater on DHAM than CHAM (p=0.018) and TCP (p=0.014) and on DDHAM than CHAM (p=0.022) and TCP (p=0.017). There was no significant difference in IL-8 expression between DHAM and DDHAM (p=1.000) or between CHAM and TCP (p=0.999).

TNFa. Although the expression of TNFα at 24 h did not vary significantly by stimulation condition and AM p=0.194), there were main effects for stimulation condition (p=0.001) and AM (p<0.001) (FIG. 13D). The overall expression of TNFa significantly increased with stimulation. Post-hoc analyses revealed that overall TNFa expression was significantly greater on DDHAM than CHAM (p<0.001) and TCP (p<0.001) and on DHAM than CHAM (p=0.022) and TCP (p=0.024). There was no significant difference in TNFa expression between DDHAM and DHAM (p=0.095) or between CHAM and TCP (p=1.000).

These results indicate that at 24 h, the presence of DDHAM and DHAM stimulated the expression of GM-CSF, IL-6, IL-8, and TNF-α in HCECs more than cells on CHAM or TCP.

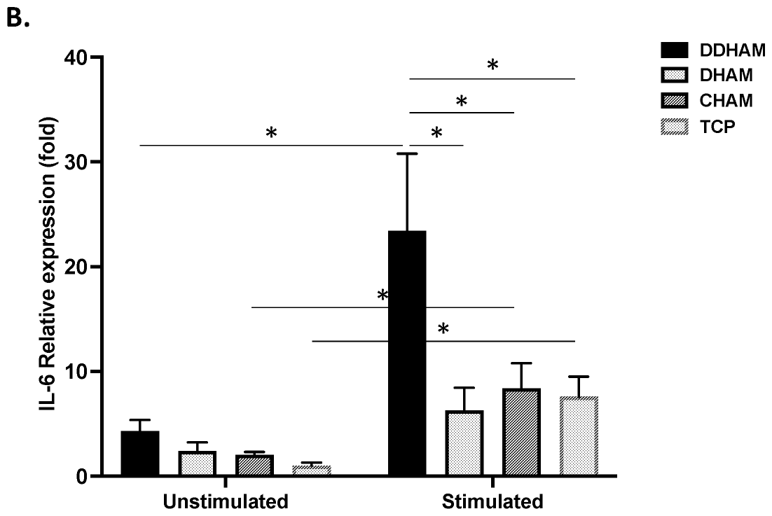

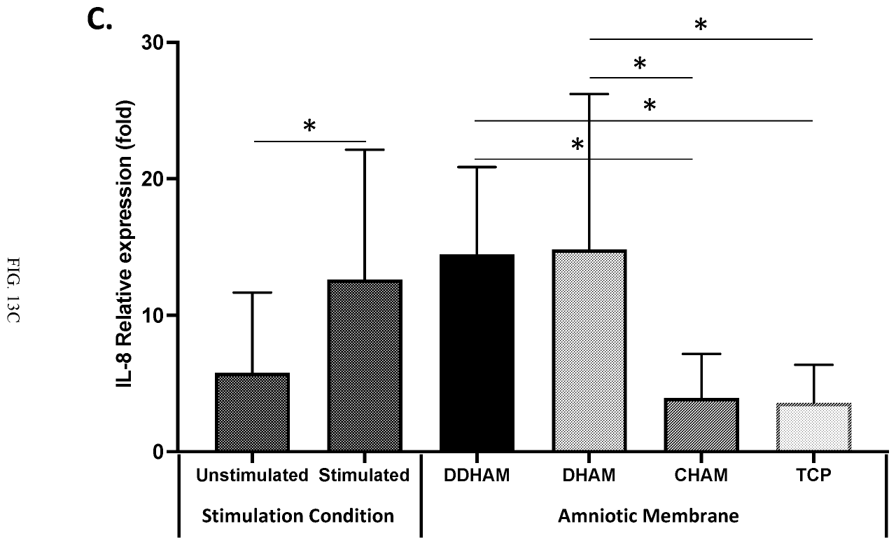

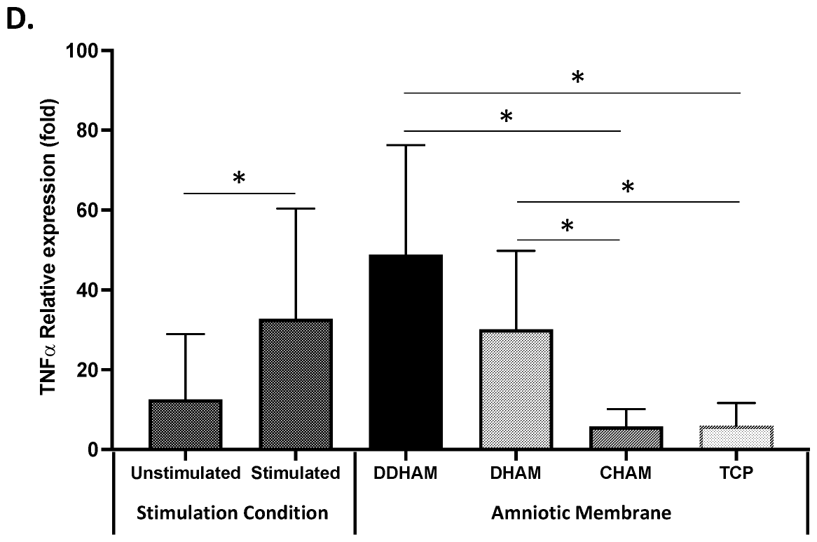

Gene expression of inflammatory cytokines in HCECs over time: The inflammatory response is a dynamic process. The expression of cytokines at different time points indicates the stage in the wound healing process. To evaluate the expression of cytokines over a 72-h time course, the expression of each cytokine was analyzed at 24 h intervals (FIGS. 14A-14D).

There were no significant changes across time in the expression of GM-CSF in the stimulated condition for DDHAM (p=0.206), DHAM (p=0.078), or CHAM (p=0.215) (FIG. 14A). TCP was an exception with significant changes across time in the expression of GM-CSF in the stimulated condition (p<0.001). The expression of GM-CSF on TCP across time significantly increased from 24 to 72 h (p<0.001) and from 48 to 72 h (p<0.001). GM-CSF expression on TCP remained similar from 24 to 48 h (p=0.700).

IL-6. There were statistically significant changes in the expression of IL-6 in the stimulated condition across time on DDHAM (p=0.007), DHAM (p<0.001), CHAM (p<0.001), and TCP (p=0.002) (FIG. 14B). Comparing the expression of IL-6 on DDHAM showed significant declines from 24 to 72 h (p=0.007) and from 48 to 72 h (p=0.021). IL-6 expression on DDHAM remained similar from 24 to 48 h (p=0.623). Comparing the expression of IL-6 on DHAM across time showed a significant increase from 24 to 48 h (p=0.003) and then a significant decrease from 48 to 72 h (p<0.001). IL-6 expression on DHAM remained similar from 24 to 72 h (p=0.321). Comparing the expression of IL-6 on CHAM across time showed significant declines from 24 to 48 h (p<0.001) and from 24 to 72 h (p<0.001). IL-6 expression on CHAM was non-detectable at both 48 and 72 h. The expression of IL-6 on TCP across time showed a significant increase from 24 to 48 h (p=0.008) and then a significant decline from 48 to 72 h (p=0.002). IL-6 expression on TCP remained similar from 24 to 72 h (p=0.407).

IL-8. Although there were statistically significant changes in the expression of IL-8 in the stimulated condition across time on CHAM (p=0.024) and TCP (p<0.001), IL-8 expression remained similar across time on DDHAM (p=0.179) and DHAM (p=0.282) (FIG. 14C). The expression of IL-8 on CHAM significantly increased from 24 to 72 h (p=0.040) and from 48 to 72 h (p=0.033). IL-8 expression on CHAM remained similar from 24 to 48 h (p=0.984). Like CHAM, the expression of IL-8 on TCP significantly increased from 24 to 72 h (p<0.001) and from 48 to 72 h (p<0.001). IL-8 expression on TCP remained similar from 24 to 48 h (p=0.071).

TNF-α. Although there were statistically significant changes in the expression of TNF-α in the stimulated condition across time on DHAM (p<0.001) and TCP (p=0.005), TNF-α expression remained similar across time on DDHAM (p=0.125) and CHAM (p=0.519) (FIG. 14D). The expression of TNF-α on DHAM across time significantly increased from 24 to 48 h (p=0.009) and significantly declined from 24 to 72 h (p=0.048), and from 48 to 72 h (p<0.001). In addition, the expression of TNF-α on TCP across time showed significant increases from 24 to 48 h (p=0.035) and from 24 to 72 h (p=0.004). TNF-α expression on TCP remained similar from 48 to 72 h (p=0.201).

The changes in relative mRNA levels across time showed different trends for different AMs and cytokines. While the expression levels increased over time in cells cultured on TCP, the expression of such cytokines showed a trend of decline in cells cultured on DDHAM.

Clinical Case Study: An 87-year-old female presented with a chief complaint of left eye deterioration, occurring over the previous few months. She reported difficulty seeing small print, due to discomfort and a foreign body sensation with prolonged reading. Her medical history was significant for dry eye syndrome, primary open-angle glaucoma, epiretinal membrane and macular drusen in both eyes. Supportive treatments included lubricant eye drops, hyperosmotic agents, and bandage contact lenses. Her ophthalmic surgical history consisted of cataract extraction in both eyes and YAG laser capsulotomy in both eyes. Upon examination, epithelial and sub-epithelial scarring in map/dot configuration was noted. Based on her presentation, history, and careful examination of the cornea, the patient was diagnosed with anterior basement membrane dystrophy (ABMD). With the patient's consent, the decision was made to treat the anterior basement membrane dystrophy surgically, using DDHAM as a substrate to repopulate the anterior corneal surface with normal Bowman's membrane (i.e., epithelium and epithelial basement membrane).

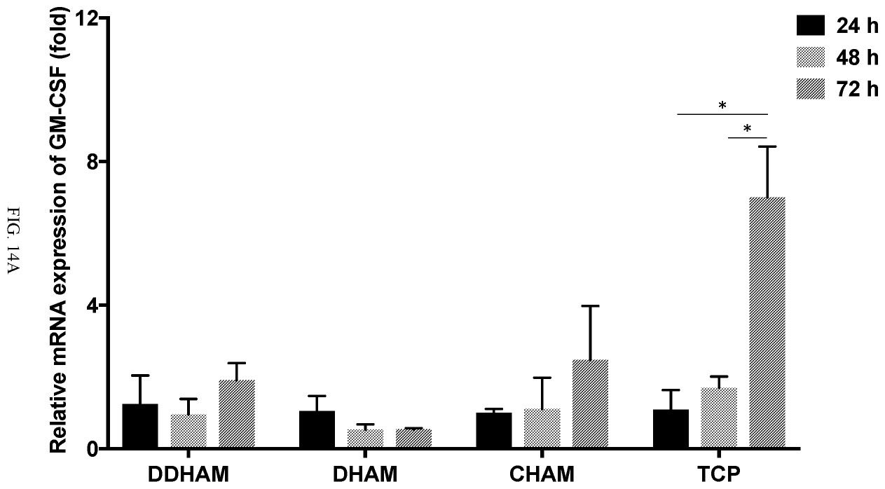

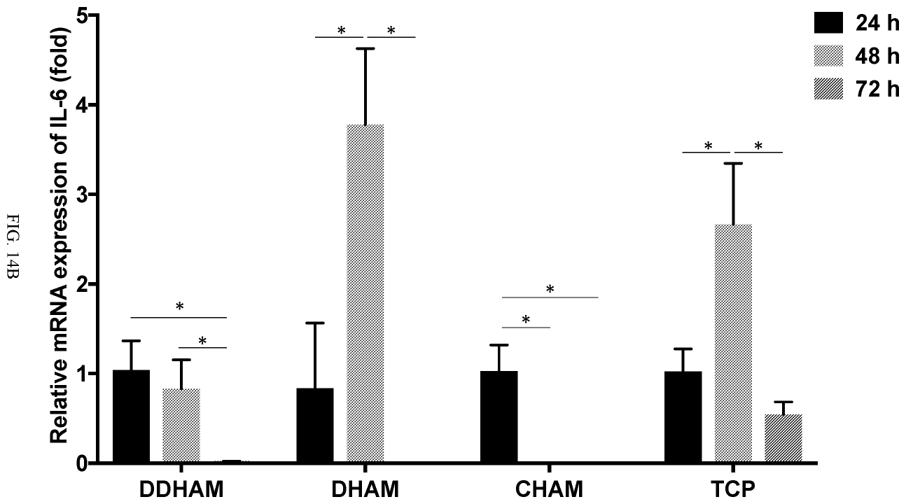

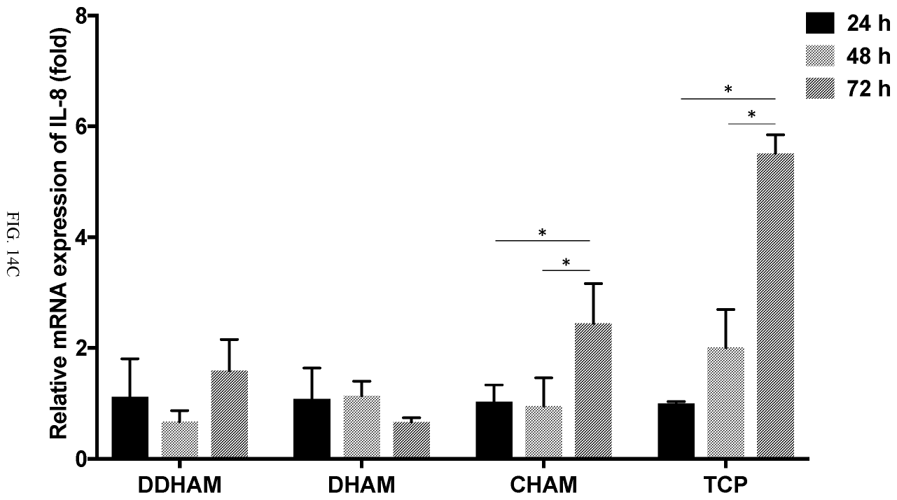

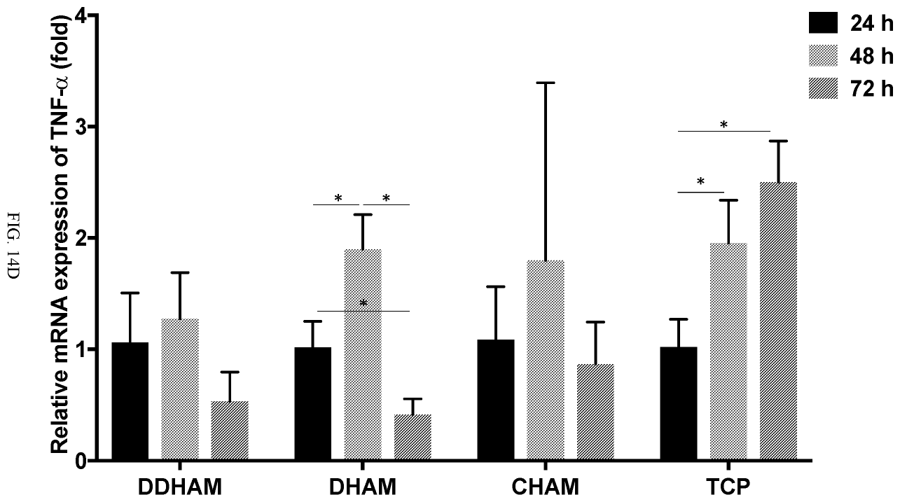

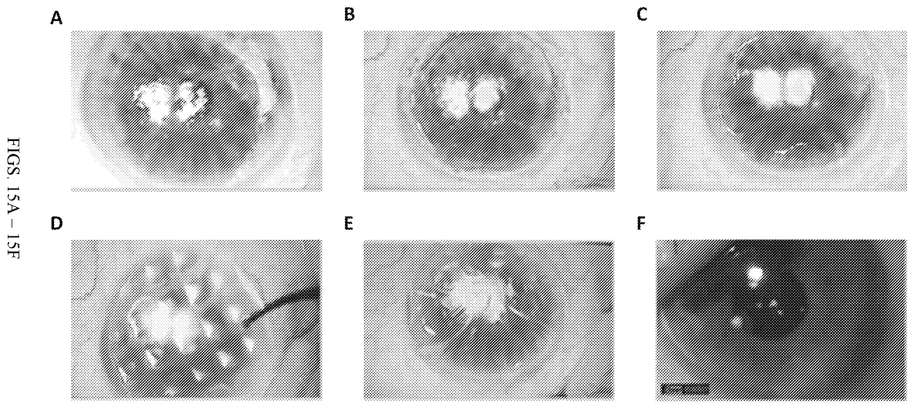

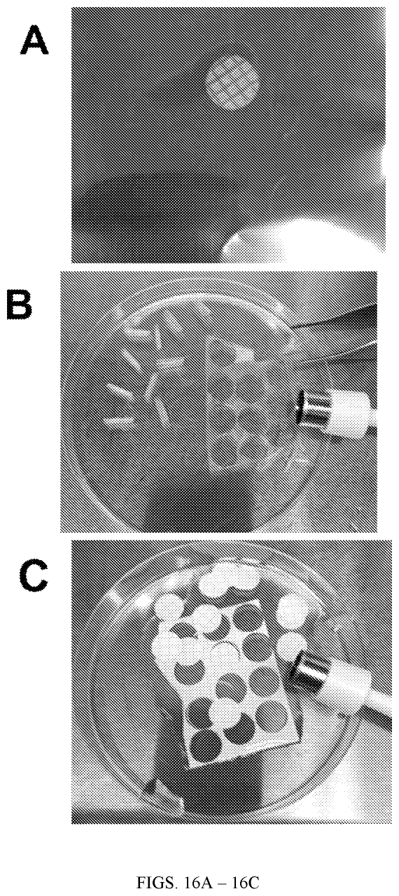

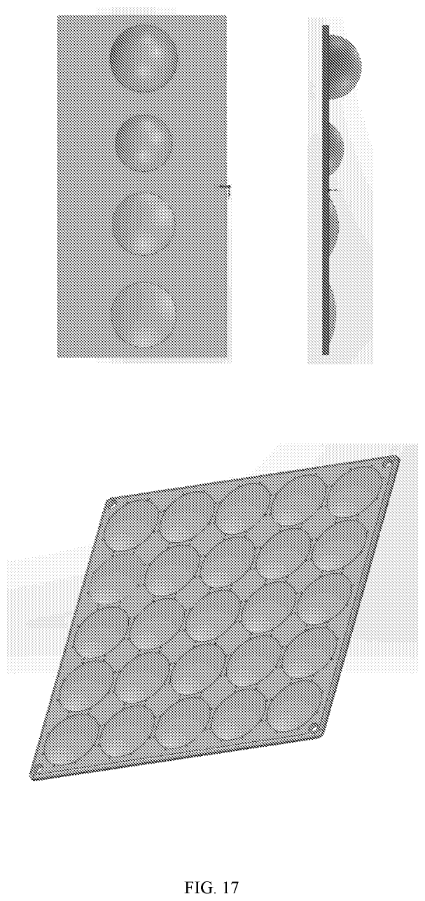

Debridement of the corneal epithelium and Bowman's membrane and placement of an AM (without sutures) was performed as an outpatient procedure. A local anesthetic was applied, and the irregular surface epithelium was visualized (FIG. 15A). A diamond burr was used to remove all abnormal, loose corneal epithelium (FIG. 15B) as well as the underlying sub-epithelial scarring and ABMD debris gently and uniformly (FIG. 15C). The epithelial surface was then rinsed with balanced salt solution. The DDHAM was carefully placed over the debrided membrane (FIG. 15D) and covered with a bandage contact lens to help with discomfort and healing (FIG. 15E). Postoperatively, the patient was instructed to use a steroid/antibiotic drop 4 times per day for 10 days, which was slowly tapered over 6 weeks. She was seen postoperatively at 1 week, 2 weeks, 1 month, and 2 months. The patient reported improved comfort in activities of daily living almost immediately. At the 1-month postoperative visit, the graft had fully dissolved into the tissue and no remnants were visible. The corneal surface was smooth and recognizable as normal (FIG. 15F).

### Discussion

The structure of the AM basement membrane is hypothesized to promote epithelialization on the ocular surface. The collagen composition closely resembles that of the conjunctiva and cornea, making the AM a suitable substrate for the growth of epithelial cells. The AM promotes the growth of corneal epithelium through four proposed mechanisms (Malhotra & Jain, 2014; Walkden et al. 2020): 1) the facilitation of epithelial cell migration (Meller et al. 2002; Meller et al. 1999), 2) the reinforcement of basal epithelial cell adhesion (Keene et al. 1987, Sonnenberg et al. 1991, Terranova et al. 1987), 3) the promotion of epithelial cell differentiation (Guo et al. 1989; Streuli et al. 1991: Kurpakus et al. 1992), and 4) the prevention of apoptosis (Boudreau et al. 1996; Boudreau et al. 1995). Although there is evidence that the stromal surface can support epithelial cell growth (Seitz et al. 2006), epithelialization is believed to occur preferentially on the basement membrane (Hu et al. 2003). However, most of the existing research is limited to cryopreserved AMs, making it unclear whether these findings are applicable to differently processed AMs.

Different processing methodologies have the potential to alter the cellular content and structure of the AM with the potential to impact the functional characteristics of ECM (Gholipourmalekabadi et al. 2015). Previous work has demonstrated significant differences in composition and ultrastructure between DDHAM and CHAM (Lim et al. 2010). Although cryopreservation is one of the most widely used preservation techniques, it has some disadvantages, namely, impacting the viability and proliferative capacity of cells as well as the need to be shipped and stored at −80° C. (Kruse et al. 2000). Therefore, the present study sought to compare how sidedness and different methods of sterilization, preservation, and decellularization impact HCEC adhesion, viability, and migration. As indicated in previous reports (Bhatia et al. 2007), the authors postulate that an ideal ocular AM requires the removal of cells, DNA, cellular debris, and residual growth factors and cytokines as well as adequate preservation of the native ECM architecture and bioactive components to prevent an inflammatory response and promote dynamic interactions between the ECM and host cells. The present study results support our hypothesis by demonstrating that DDHAM is a fully decellularized AM, whereas DHAM and CHAM contain residual cells and DNA. DDHAM was then found to best support the cellular activities of HCECs. In addition, the presence of DDHAM enhances an initial inflammatory response and prevents a prolonged inflammatory response in HCECs under an in vitro inflammatory condition.

Staining confirms the absence of cells and nuclei in DDHAM. Previous research documents that the biological effectiveness of AMs in ophthalmology is facilitated by its ECM, rather than cells preserved in the AM (Dua et al. 2004; Kubo et al. 2001; Kruse et al. 2000). In decellularized AM, the ECM is presumed to serve as a physical conduit for cellular infiltration, whereby the host cells and ECM interact to provide the necessary biochemical stimulus to activate a healing response (Bhatia et al. 2007). Therefore, as a preliminary step, staining was performed on each of the three AMs to visualize the cellular content and structure. Both immunofluorescent and H&E staining confirmed complete decellularization and the absence of nuclei in DDHAM, whereas both DHAM and CHAM showed nuclear content, remnants in DHAM and the presence of cells in CHAM.

Stromal side of DDHAM best supports the cellular activities of HCEC. The results from this in vitro investigation suggest that the stromal side of DDHAM best supports HCEC activity. Sidedness did not impact HCEC adhesion on DDHAM or CHAM, but HCEC adhesion was significantly lower on the epithelial side of DHAM. The difference in cellular adhesion between DDHAM and DHAM, two dehydrated AMs, suggests that the removal of cellular components, DNA, growth factors and cytokines provides a more cell-friendly environment, supporting the attachments of HCECs.

When examined across time, cell viability was found to decrease for all sidedness and AM combinations, except for the stromal side of DDHAM. On the stromal side of DDHAM, cell viability increased from day 1 to day 4. The specific cause of the overall decrease in cell viability is not clear. The presence of amnion cells (cryopreserved or dried) in the CHAM or DHAM may inhibit the ability of these AMs to support corneal cell proliferation. Although it has been reported previously that decellularized amniotic membrane is a better substrate than fresh amnion for corneal epithelial cells (Koizumi et al. 2000), these results suggest that sidedness may also be a factor. This study found that the stromal side of DDHAM is the most compatible substrate for the growth of HCECs, whereas neither the epithelial or stromal sides of CHAM and DHAM appear to consistently support their adhesion and growth.

These findings are further supported by staining. On day four, DDHAM demonstrated the most homogeneous growth pattern of HCECs (FIG. 10). As indicated by actin staining, the morphology and organization of cells on DDHAM is similar to the morphology of corneal epithelial cells in situ (FIGS. 11A and 11B) (Sosnová-Netuková et al. 2007). These observations suggest orderly growth on the AM. Conversely, the growth pattern on DHAM appears disorganized, and it remains unclear whether the HCECs on CHAM are viable or existent. It has been well-established that when cells are stressed, they change phenotype (Kumar et al. 2013). While there are many factors to consider, these results suggest that the differences in the dehydration, cryopreservation, and decellularization processes may impact how the cells interact with the membrane, specifically in terms of cell adhesion and cell viability.

Differently processed AMs may also affect the release of factors from epithelial cells cultured on them. To evaluate the effect of AM alone on the migration of HCECs, the present study tested the conditioned media from three different AMs with and without cells and found that HCECs migrated more in the presence of conditioned media with cells than without cells on DDHAM and DHAM. However, there was no difference in HCEC migration in the presence of conditioned media with or without cells on CHAM or on the control. These findings suggest that the factors released by the cells promote cell migration beyond that of the AM (i.e., DDHAM and DHAM) alone. In addition, the migration of HCEC in the presence of conditioned media from cells on DDHAM and from cells on DHAM were comparable, and both were significantly greater than cells on CHAM. One possible explanation of this finding is that there were fewer cells on CHAM when the conditioned medium was collected. With fewer cells, the stimulatory effect of the conditioned media may be lower, resulting in less migration in the presence of conditioned medium from cells on CHAM. Additionally, the migration of HCECs in the presence of conditioned media with cells was significantly greater on all three AMs than the medium control. Collectively, these findings suggest that factors released from cells and AMs promote cell migration and that the factors released vary by AM, resulting in more HCEC migration on DDHAM and DHAM than CHAM. Additional studies are needed to determine the identity and sources of these factors.

An additional independent experiment was conducted to determine whether sidedness influences HCEC migration. The experiment followed the same methodology as described in the ‘Conditioned Media for Migration Assay’ and ‘Scratch Wound Migration Assay’ sections. In this experiment, however, the migration of HCECs in the presence of conditioned media was evaluated on both the stromal and epithelial sides of the AMs. The results from this experiment confirmed that there is no difference in HCEC migration in the presence of conditioned media from cells on the epithelial or on stromal sides of the AMs (p=0.407; data not shown).

Traditionally, the AM is placed as a graft with epithelial side up to promote epithelialization over a defect. Both DHAM and CHAM have this clinical applicability due to their sidedness. However, DDHAM is manufactured with the stromal side facing out to interface with the ocular surface regardless of orientation. The results from this in vitro study demonstrated that HCEC activity was highest on the stromal side of DDHAM, thus supporting its clinical applicability as a graft. Moreover, the included case study demonstrated the successful application of DDHAM to treat anterior basement membrane dystrophy. One-month postoperatively, the corneal surface was smooth and recognizable as normal, which could be indicative of progressing re-epithelialization. However, histology at additional time points is necessary to demonstrate reorganization and remodeling of the corneal epithelium, its basement membrane, and Bowman's layer. While encouraging, additional, in vivo investigations with a larger sample size are needed to evaluate DDHAM more fully as well as its ability to promote epithelialization on the ocular surface.

DDHAM supports an initial inflammatory response, followed by a declining trend across time.

The anti-inflammatory properties of AM have been well-documented (Sharma et al. 2016; Tabatabaei et al. 2017; Tandon et al. 2011). Based on in-vitro research, AMs reduce the expression of growth factors and pro-inflammatory cytokines from the damaged ocular tissue (Solomon et al. 2001), while also trapping inflammatory cells and inducing apoptosis (Dua et al. 2004; Shimmura et al. 2001). Therefore, the secondary aim of this investigation was to evaluate the inflammatory response of HCECs on different AMs. This was accomplished by examining the immediate mRNA expression as well as trends across time. Given their known roles in corneal wound healing, the pro-inflammatory cytokines, GM-CSF, IL-6, IL-8, and TNF-α were selected to assess the inflammatory response of HCECs.

GM-CSF is recognized as both an inflammatory (van Nieuwenhuijze et al. 2013) and immunoregulatory cytokine (Parmiani et al. 2007) with its effects dependent on dose and context (Bhattacharya et al. 2015; Parmiani et al. 2007; Shachar and Karin 2013). This multipotent cytokine has been recognized for having important roles in inflammation and wound healing and more specifically has a proven ability to enhance corneal wound healing both in vitro and in vivo (Rho et al. 2015). IL-6, IL-8, and TNF-α are more traditional pro-inflammatory cytokines. In addition to regulating the inflammatory and immune responses, IL-6 has been shown to facilitate corneal wound healing in vitro and in vivo (Arranz-Valsero et al. 2014; Ebihara et al. 2011; Nishida et al. 1992; Hafezi et al. 2018). IL-8 is a corneal factor that induces neovascularization and is thought to modulate wound healing (Strieter et al. 1992; Koch et al. 1992). Lastly, TNF-α is involved in the corneal inflammatory response and wound healing following corneal injuries (Wang et al. 2020; Yang et al. 2019).

In the present study, there was a higher expression of inflammatory cytokines (i.e., IL-6, IL-8, TNF-α) in cells cultured on DDHAM in the first 24 h, followed by a declining trend across time. These observations suggest that the presence of DDHAM may promote an initial inflammatory response and prevent a prolonged inflammatory response in HCEC cells, which may be advantageous in a wound healing environment. However, additional in vivo research is needed to evaluate these findings more fully.

The AM is used for ocular surface reconstruction to treat a wide variety of ocular pathologies, including corneal surface disorders with and without limbal stem cell deficiency (Maharajan et al. 2007; Sangwan et al. 2012), reconstruction of the conjunctival surface (e.g., pterygium removal [Rock et al. 2019; Akbari et al. 2017]), as a carrier for ex vivo expansion of limbal epithelial cells (Rama et al. 2010; Shortt et al. 2009), glaucoma (Sheha et al. 2008), neoplasia (Agraval et al. 2017), sclera melts and perforations (Hanada et al. 2001; Ma et al. 2002), among others. Given its potential to enhance healing, integrate with host tissue, and avoid a foreign body response, decellularized AM has gained increasing interest in recent years (Gholipourmalekabadi et al. 2015; Fenelon et al. 2019; Lim et al. 2010; Koizumi et al. 2000; Salah et al. 2018; Fransisco et al. 2016; Gholipourmalekabadi et al. 2016; Taghiabadi et al. 2015). Adequate preservation of the ECM in decellularized AM has been shown to improve the interaction of various cell types within the AM, with evidence of improved cell adhesion, proliferation, and differentiation (Fenelon et al. 2019; Koizumi et al. 2000; Salah et al. 2018: Fransisco et al. 2016; Gholipourmalekabadi et al. 2016; Taghiabadi et al. 2015). Moreover, and perhaps most importantly, decellularized AM has been shown to integrate into biological tissue with low immunogenicity (Fenelon et al. 2019; Fransisco et al. 2016; Gholipourmalekabadi et al. 2016).

AmbioDry™ is a single-layer AM that has been low-dose electron beam sterilized and preserved through dehydration with the epithelial layer mechanically eliminated (Hovanesian, 2012). Although the product is no longer available, much can be garnered from the scientific evaluation of this DDHAM product (Memarzadeh et al. 2008: Chuck et al. 2004). Memarzadeh et al. demonstrated its ability to act as an effective conjunctival autograft in preventing pterygium recurrence (Memarzadeh et al. 2008). Additionally, a biomechanical research study confirmed that this DDHAM maintains desirable elastic characteristics when rehydrated, making it an easy-to-manipulate tissue for ocular surface reconstruction (Chuck et al. 2004). Despite distinct differences between AmbioDry™ and Biovance®3L Ocular, such as Biovance® 3L Ocular's unique three-layer design as well as its complete removal of cells and associated growth factors (Bhatia et al. 2007), these previous publications provide additional insight into DDHAM products and their clinical application in ophthalmology.

While the results from the present study are encouraging, there are several limitations. First and foremost, findings from in vitro investigations do not directly translate to clinical application. A superb compatibility with ocular epithelial cells does not necessarily equate to clinical improvements in ocular wound healing. Unlike this in vitro study, many types of cells exist and interact with each other in tissues in vivo. The cellular behavior of one cell type does not necessarily represent the responses of the tissue. Despite these limitations, however, this study is unique in its comparison of ocular cell activity and inflammatory response on three commercially available AMs. Furthermore, this study is the first to demonstrate the effect of AM sidedness on cellular activities.

### Conclusion

Overall, DDHAM was shown to support better HCEC functionality in vitro, which may suggest greater ocular cell compatibility in vivo. Additional research is warranted to evaluate the wound healing response of DDHAM as well as its clinical application and outcomes.

### REFERENCES

Leal-Marin S, Kern T, Hofmann N, Pogozhykh O, Framme C, Börgel M, Figueiredo C, Glasmacher B, Gryshkov O. Human Amniotic Membrane: A review on tissue engineering, application, and storage. J Biomed Mater Res B Appl Biomater. 2021: 109:1198-1215.

Walkden A. Amniotic membrane transplantation in Ophthalmology: an updated perspective. Clin Ophthalmol. 2020:14:2057-2072.

Liu J, Li L, Li X. Effectiveness of Cryopreserved Amniotic Membrane Transplantation in Corneal Ulceration: A Meta-Analysis. Cornea. 2019 Apr:38:454-462.

Malhotra C, Jain A K. Human amniotic membrane transplantation: different modalities of its use in ophthalmology. World J Transplant. 2014:4: 111-121.

Meller D, Pauklin M, Thomasen H, Westekemper H, Steuhl K P. Amniotic membrane transplantation in the human eye. Dtsch Arztebl Int. 2011:108:243-248.

Fernandes M, Sridhar M S, Sangwan V S, Rao G N. Amniotic membrane transplantation for ocular surface reconstruction. Cornea. 2005:24:643-653.

Shayan Asl N, Nejat F, Mohammadi P, Nekoukar A, Hesam S, Ebrahimi M, Jadidi K. Amniotic membrane extract eye drop promotes limbal stem cell proliferation and corneal epithelium healing. Cell J. 2019:20:459-468.

Meller D, Pires R T, Tseng S C. Ex vivo preservation and expansion of human limbal epithelial stem cells on amniotic membrane cultures. Br J Ophthalmol. 2002:86:463-471.

Meller D, Tseng S C. Conjunctival epithelial cell differentiation on amniotic membrane. Invest Ophthalmol Vis Sci. 1999:40:878-886.

Sharma N, Singh D, Maharana P K, Kriplani A, Velpandian T, Pandey R M, Vajpayee R B. Comparison of amniotic membrane transplantation and umbilical cord serum in acute ocular chemical burns: a randomized controlled trial. Am J Ophthalmol. 2016;168:157-163.

Tabatabaei S A, Soleimani M, Behrouz M J, Torkashvand A, Anvari P, Yaseri M. A randomized clinical trial to evaluate the usefulness of amniotic membrane transplantation in bacterial keratitis healing. Ocul Surf. 2017:15:218-226.

Tandon R, Gupta N, Kalaivani M, Sharma N, Titiyal J S, Vajpayee R B. Amniotic membrane transplantation as an adjunct to medical therapy in acute ocular burns. Br J Ophthalmol. 2011:95:199-204.

Niknejad H, Peirovi H, Jorjani M, Ahmadiani A, Ghanavi J, Seifalian A M. Properties of the amniotic membrane for potential use in tissue engineering. Eur Cells Mater. 2008:15:88-99.

Tseng S C, Li D Q, Ma X. Suppression of transforming growth factor-beta isoforms, TGF-beta receptor type II, and myofibroblast differentiation in cultured human corneal and limbal fibroblasts by amniotic membrane matrix. J Cell Physiol. 1999:179:325-335.

Lee S B, Li D Q, Tan D T, Meller D C, Tseng S C. Suppression of TGF-beta signaling in both normal conjunctival fibroblasts and pterygial body fibroblasts by amniotic membrane. Curr Eye Res. 2000;20:325-334.

Hao Y, Ma D H, Hwang D G, Kim W S, Zhang F. Identification of antiangiogenic and antiinflammatory proteins in human amniotic membrane. Cornea. 2000: 19:348-352.

Mamede A C, Botelho M F. Amniotic membrane: origin, characterization and medical applications. Mamede A C, Botelho M F, editors. New York, N.Y.: Springer: 2015.

Tehrani F A, Peirovi H, Niknejad. Determination of antibacterial effect of the epithelial and mesenchymal surfaces of amniotic membrane on escherichia coli, staphylococcus aureus and pseudomonas aeruginosa. Qom Univ Med Sci J. 2013:7:12-22.

Sangwan V S, Basu S. Antimicrobial properties of amniotic membrane. Br J Ophthalmol. 2011:95:1.

Kjaergaard N, Hein M, Hyttel L, Helmig R B, Schonheyder H C, Uldbjerg N, Madsen H. Antibacterial properties of human amnion and chorion in vitro. Eur J Obstet Gynecol Reprod Biol. 2001:94:224-229.

Kjaergaard N, Helmig R B, Schonheyder H C, Uldbjerg N, Hansen E S, Madsen H. Chorioamniotic membranes constitute a competent barrier to group b streptococcus in vitro. Eur J Obstet Gynecol Reprod Biol. 1999:83:165-169.

Inge E, Talmi Y P, Sigler L, Finkelstein Y, Zohar Y. Antibacterial properties of human amniotic membranes. Placenta. 1991: 12:285-288.

Dua H S, Gomes J A, King A J, Maharajan V S. The amniotic membrane in ophthalmology. Surv Ophthalmol. 2004:49:51-77.

Shimmura S, Shimazaki J, Ohashi Y, Tsubota K. Antiinflammatory effects of amniotic membrane transplantation in ocular surface disorders. Cornea. 2001:20:408-413.

Hu D J, Basti A, Wen A, Bryar P J. Prospective comparison of corneal re-epithelialization over the stromal and basement membrane surfaces of preserved human amniotic membrane. ARVO Annual Meeting Abstract, 2003.

Seitz B, Resch MD, Schlötzer-Schrehardt U, Hofmann-Rummelt C, Sauer R, Kruse F E. Histopathology and ultrastructure of human corneas after amniotic membrane transplantation. Arch Ophthalmol: 2006;124:1487-1490.

von Versen-Hoynck F, Syring C, Bachmann S, Möller DE. The influence of different preservation and sterilisation steps on the histological properties of amnion allografts—light and scanning electron microscopic studies. Cell Tissue Bank. 2004:5:45-56.

Lim L S, Poh R W, Riau A K, Beuerman R W, Tan D, Mehta J S. Biological and ultrastructural properties of acelagraft, a freeze-dried γ-irradiated human amniotic membrane. Arch Ophthalmol. 2010:128:1303-1310.

Tehrani F D, Firouzeh A, Shabani I, Shabani A. A review on modifications of amniotic membrane for biomedical applications. Front Bioeng Biotechnol. 2021:13:8:606982.

Gholipourmalekabadi M, Mozafari M, Salehi M, et al. Development of a cost-effective and simple protocol for decellularization and preservation of human amniotic membrane as a soft tissue replacement and delivery system for bone marrow stromal cells. Adv Healthc Mater. 2015:4:918-926.

Keane T J, Londono R, Turner N J, Badylak S F. Consequences of ineffective decellularization of biologic scaffolds on the host response. Biomaterials. 2012:33:1771-1781.

Seif-Naraghi S B, Singelyn J M, Salvatore M A, Osborn K G, Wang J J, Sampat U, Kwan O L, Strachan G M, Wong J, Schup-Magoffin P J, Braden R L, Bartels K, DeQuach J A, Preul M, Kinsey A M, DeMaria A N, Dib N, Christman K L. Safety and efficacy of an injectable extracellular matrix hydrogel for treating myocardial infarction. Sci Transl Med. 2013:5:173ra25.

Aamodt J M, Grainger D W. Extracellular matrix-based biomaterial scaffolds and the host response. Biomaterials. 2016:86:68-82.

Balestrini J L, Gard A L, Liu A, et al. Production of decellularized porcine lung scaffolds for use in tissue engineering. Integr Biol (Camb). 2015:7:1598-1610.

Bhatia M, Pereira M, Rana H, et al. The mechanism of cell interaction and response on decellularized human amniotic membrane: implications in wound healing. Wounds. 2007: 19:207-217.

Rodríguez-Ares M T, López-Valladares M J, Touriño R, Vieites B, Gude F, Silva M T, Couceiro J. Effects of lyophilization on human amniotic membrane. Acta Ophthalmol. 2009:87:396-403.

Keene D R, Sakai L Y, Lunstrum G P, Morris N P, Burgeson R E. Type VII collagen forms an extended network of anchoring fibrils. J Cell Biol. 1987:104:611-621.

Sonnenberg A, Calafat J, Janssen H, Daams H, van der Raaij-Helmer L M, Falcioni R, Kennel S J, Aplin J D, Baker J, Loizidou M, et al. Integrin alpha 6/beta 4 complex is located in hemidesmosomes, suggesting a major role in epidermal cell-basement membrane adhesion. J Cell Biol. 1991:113(4): 907-917.

Terranova V P, Lyall R M. Chemotaxis of human gingival epithelial cells to laminin. A mechanism for epithelial cell apical migration. J Periodontol. 1986;57(5):311-317.

Guo M, Grinnell F. Basement membrane and human epidermal differentiation in vitro. J Invest Dermatol. 1989:93:372-378.

Kurpakus M A, Stock E L, Jones J C. The role of the basement membrane in differential expression of keratin proteins in epithelial cells. Dev Biol. 1992: 150:243-255.

Streuli C H, Bailey N, Bissell M J. Control of mammary epithelial differentiation: basement membrane induces tissue-specific gene expression in the absence of cell-cell interaction and morphological polarity. J Cell Biol. 1991:115:1383-95.

Boudreau N, Werb Z, Bissell M J. Suppression of apoptosis by basement membrane requires three-dimensional tissue organization and withdrawal from the cell cycle. Proc Natl Acad Sci USA. 1996:93:3509-3513.

Boudreau N, Sympson C J, Werb Z, Bissell M J. Suppression of ICE and apoptosis in mammary epithelial cells by extracellular matrix. Science. 1995:267:891-893.

Kubo M, Sonoda Y, Muramatsu R, Usui M. Immunogenicity of human amniotic membrane in experimental xenotransplantation. Invest Ophthal Vis Sci. 2001: 42:1539-1546.

Kruse F E, Joussen A M, Rohrschneider K, et al. Cryopreserved human amniotic membrane for ocular surface reconstruction. Graefe's Arch Clin Exp Ophthalmol. 2000:238: 68-75.

Koizumi N, Fullwood N J, Bairaktaris G, Inatomi T, Kinoshita S, Quantock A J. Cultivation of comeal epithelial cells on intact and denuded human amniotic membrane. Invest Ophthalmol Vis Sci. 2000:41:2506-2513.

Sosnová-Netuková M, Kuchynka P, Forrester J V. The suprabasal layer of corneal epithelial cells represents the major barrier site to the passive movement of small molecules and trafficking leukocytes. Br J Ophthalmol. 2007:91:372-378.

Kumar V, Abbas A K, Aster J C. Cell Injury, Cell Death, and Adaptations. In: Kumar V, Abbas A K, Aster J C, ed. Robins Basic Pathology. Philadelphia: Elsevier Saunders, 2013:1-28.

Solomon A, Rosenblatt M, Monroy D, Ji Z, Pflugfelder S C, Tseng S C. Suppression of Interleukin 1 alpha and Interleukin 1 beta in the human limbal epithelial cells cultured on the amniotic membrane stromal matrix. Br J Ophthalmol. 2001:85: 444-449.

van Nieuwenhuijze A, Koenders M, Roeleveld D, Sleeman M A, van den Berg W, Wicks I P. GM-CSF as a therapeutic target in inflammatory diseases. Mol Immunol. 2013:56:675-682.

Parmiani G, Castelli C, Pilla L, Santinami M, Colombo MP, Rivoltini L. Opposite immune functions of GM-CSF administered as vaccine adjuvant in cancer patients. Ann Oncol. 2007:18:226-232.

Bhattacharya P, Budnick I, Singh M, Thiruppathi M, Alharshawi K, Elshabrawy H, Holterman M J, Prabhakar B S. Dual Role of GM-CSF as a Pro-Inflammatory and a Regulatory Cytokine: Implications for Immune Therapy. J Interferon Cytokine Res. 2015:35:585-599.

Shachar I, Karin N. The dual roles of inflammatory cytokines and chemokines in the regulation of autoimmune diseases and their clinical implications. J Leukoc Biol. 2013;93:51-61.

Rho C R, Park M Y, Kang S. Effects of granulocyte-macrophage colony-stimulating (GM-CSF) factor on corneal epithelial cells in corneal wound healing model. PLOS One. 2015:10:e0138020.

Hafezi F, Gatzioufas Z, Angunawela R, Ittner L M. Absence of IL-6 prevents corneal wound healing after deep excimer laser ablation in vivo. Eye (Lond). 2018:32:156-157.

Arranz-Valsero I, Soriano-Romani L, García-Posadas L, López-García A, Diebold Y. IL-6 as a corneal wound healing mediator in an in vitro scratch assay. Exp Eye Res. 2014:125:183-192.

Ebihara N, Matsuda A, Nakamura S, Matsuda H, Murakami A. Role of the IL-6 classic- and trans-signaling pathways in corneal sterile inflammation and wound healing. Invest Ophthalmol Vis Sci. 2011:52:8549-8557.

Nishida T, Nakamura M, Mishima H, Otori T, Hikida M. Interleukin 6 facilitates corneal epithelial wound closure in vivo. Arch Ophthalmol. 1992: 110: 1292-1294.

Strieter R M, Kunkel S L, Elner V M, Martonyi C L, Koch A E, Polverini P J, Elner S G. Interleukin-8. A corneal factor that induces neovascularization. Am J Pathol. 1992:141:1279-1284.

Koch A E, Polverini P J, Kunkel S L, Harlow L A, DiPietro L A, Elner V M, Elner S G, Strieter RM. Interleukin-8 as a macrophage-derived mediator of angiogenesis. Science. 1992:258:1798-1801.

Wang X, Zhang S, Dong M, Li Y, Zhou Q, Yang L. The proinflammatory cytokines IL-1β and TNF-α modulate corneal epithelial wound healing through p16Ink4a suppressing STAT3 activity. J Cell Physiol. 2020:235:10081-10093.

Yang L, Zhang S, Duan H, Dong M, Hu X, Zhang Z, Wang Y, Zhang X, Shi W, Zhou Q. Different effects of pro-Inflammatory factors and hyperosmotic stress on corneal epithelial stem/progenitor cells and wound healing in mice. Stem Cells Transl Med. 2019;8:46-57.

Sangwan V S, Basu S, MacNeil S, Balasubramanian D. Simple limbal epithelial transplantation (SLET): a novel surgical technique for the treatment of unilateral limbal stem cell deficiency. Br J Ophthalmol. 2012 Jul:96(7): 931-934.

Maharajan V S, Shanmuganathan V, Currie A, Hopkinson A, Powell-Richards A, Dua H S. Amniotic membrane transplantation for ocular surface reconstruction: indications and outcomes. Clin Exp Ophthalmol. 2007 Mar:35:140-147.

Röck T, Bramkamp M, Bartz-Schmidt K U, Röck D. A retrospective study to compare the recurrence rate after treatment of pterygium by conjunctival autograft, primary closure, and amniotic membrane transplantation. Med Sci Monit. 2019:25:7976-7981.

Akbari M, Soltani-Moghadam R, Elmi R, Kazemnejad E. Comparison of free conjunctival autograft versus amniotic membrane transplantation for pterygium surgery. J Curr Ophthalmol. 2017:29:282-286.

Rama P, Matuska S, Paganoni G, Spinelli A, De Luca M, Pellegrini G. Limbal stem-cell therapy and long-term corneal regeneration. N Engl J Med. 2010:363:147-155.

Shortt A J, Secker G A, Lomas R J, Wilshaw S P, Kearney J N, Tuft S J, Daniels J T. The effect of amniotic membrane preparation method on its ability to serve as a substrate for the ex-vivo expansion of limbal epithelial cells. Biomaterials. 2009:30:1056-65.

Sheha H, Kheirkhah A, Taha H. Amniotic membrane transplantation in trabeculectomy with mitomycin C for refractory glaucoma. J Glaucoma. 2008:17:303-307.

Agraval U, Rundle P, Rennie I G, Salvi S. Fresh frozen amniotic membrane for conjunctival reconstruction after excision of neoplastic and presumed neoplastic conjunctival lesions. Eye (Lond). 2017:31:884-889.

Hanada K, Shimazaki J, Shimmura S, Tsubota K. Multilayered amniotic membrane transplantation for severe ulceration of the cornea and sclera. Am J Ophthalmol. 2001: 131:324-331.

Ma D H, Wang S F, Su W Y, Tsai R J. Amniotic membrane graft for the management of scleral melting and corneal perforation in recalcitrant infectious scleral and comneoscleral ulcers. Cornea. 2002:21:275-283.

Fenelon M, Maurel D B, Siadous R, et al. Comparison of the impact of preservation methods on amniotic membrane properties for tissue engineering applications. Mat Sci Eng C. 2019:104: 109903.

Salah R A, Mohamed I K, El-Badri N. Development of decellularized amniotic membrane as a bioscaffold for bone marrow-derived mesenchymal stem cells: ultrastructural study. J Mol Histol. 2018:49:289-301.

Francisco J C, Correa Cunha R, Cardoso M A, Baggio Simeoni R, Mogharbel B F, Picharski GL, Silva Moreira Dziedzic D, Guarita-Souza L C, Carvalho K A. Decellularized Amniotic Membrane Scaffold as a Pericardial Substitute: An In Vivo Study. Transplant Proc. 2016:48:2845-2849.

Gholipourmalekabadi M, Sameni M, Radenkovic D, Mozafari M, Mossahebi-Mohammadi M, Seifalian A. Decellularized human amniotic membrane: how viable is it as a delivery system for human adipose tissue-derived stromal cells? Cell Prolif. 2016:49:115-121.

Taghiabadi E, Nasri S, Shafieyan S, Firoozinezhad S J, Aghdami N. Fabrication and characterization of spongy denuded amniotic membrane based scaffold for tissue engineering. Cell J. 2015:16:476-487.

Memarzadeh F, Fahd A K, Shamie N, Chuck R S. Comparison of de-epithelialized amniotic membrane transplantation and conjunctival autograft after primary pterygium excision. Eye (Lond). 2008:22:107-112.

Chuck R S, Graff J M, Bryant M R, Sweet P M. Biomechanical characterization of human amniotic membrane preparations for ocular surface reconstruction. Ophthalmic Res. 2004:36:341-348.

Hovanesian J A. History of amniotic membranes in pterygium surgery. In: Hovanesian J A, editor. Pterygium: techniques and technologies for surgical success. Thorofare: SLACK Incorporated; 2012: 65-75.

### Exaple 6

### Curved Biovance 3L Ocular

In the present example, Biovance 3L ocular is created in a curved format to better fit the cornea and eyeball.

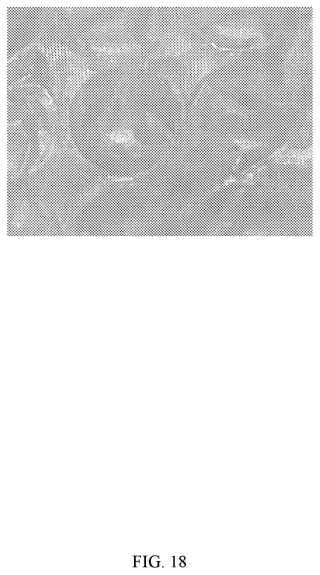

Molds were fabricated by 3D printing having different spherical radii, heights, and diameters (FIG. 17). Layered membranes are dried to the molds and carefully removed. The dried product, FIG. 18, is cut in the space between curved units.

TABLE 7
Curved biovance 3L ocular

| Spherical | | Total | Bottom | Radial | |
|---|---|---|---|---|---|
| Radius | Height | Angle | Diameter | Distance | Assumptions: |
| 5.00 | 5.00 | 90.00 | 10.00 | 15.71 | Current 2D die cutter |
| 5.00 | 2.30 | 57.32 | 8.42 | 10.00 | diameter = 10 mm |
| 8.00 | 1.50 | 35.66 | 9.33 | 9.96 | Dimensions of our |
| | | | | | first mold |
| | | | | | Average cornea |
| | | | | | spherical |
| | | | | | diameter = 16 mm |
| 11.50 | 1.07 | 24.91 | 9.69 | 10.00 | Most common |
| | | | | | sizes—10 and 12 mm |
| | | | | | Average eyeball |
| | | | | | spherical |
| | | | | | diameter = 23 mm |

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

In the claims:

1. A tissue graft product comprising a plurality of layers of extracellular matrix laminated together, wherein the extracellular matrix is derived from an amniotic membrane, and wherein the stromal side of an extracellular matrix layer is presented on both the upper and lower surfaces of the tissue graft product.

2. The tissue graft product of claim 1, wherein the product comprises three or more layers of extracellular matrix.

3. (canceled)

4. (canceled)

5. (canceled)

6. (canceled)

7. (canceled)

8. (canceled)

9. The tissue graft product of claim 1, wherein the tissue graft product is dehydrated.

10. The tissue graft product of claim 1, wherein the product comprises less than about 20%, less than about 15%, or less than about 10% water by dry weight.

11. (canceled)

12. (canceled)

13. The tissue graft product of claim 1, wherein the product comprises about 40% to about 70% total collagen by dry weight, about 45% to about 60% total collagen by weight, or about 50% to about 55% total collagen by dry weight.

14. (canceled)

15. (canceled)

16. The tissue graft product of claim 13, wherein the collagen is primarily collagen type I and collagen type III.

17. The tissue graft product of claim 1, wherein the product comprises about 8% to about 24% elastin by dry weight, about 12% to about 20% elastin by dry weight, or about 15% to about 20% elastin by dry weight.

18. (canceled)

19. (canceled)

20. The tissue graft product of claim 1, wherein the product comprises less than about 1% glycosaminoglycan by dry weight, or less than about 0.5% glycosaminoglycan by dry weight.

21. (canceled)

22. The tissue graft product of claim 1, wherein the product comprises less than about 1% fibronectin by dry weight, or less than about 0.5% fibronectin by dry weight.

23. (canceled)

24. The tissue graft product of claim 1, wherein the product comprises less than about 1% laminin by dry weight, or less than about 0.5% laminin by dry weight.

25. (canceled)

26. The tissue graft product of claim 1, wherein the amniotic membrane is a human amniotic membrane.

27. The tissue graft product of claim 26, wherein the amniotic membrane is derived from a full-term pregnancy.

28. An ocular tissue graft comprising the tissue graft product of claim 1.

29. The ocular tissue graft of claim 28, wherein the ocular tissue graft is approximately circular.

30. The ocular tissue graft of claim 28, wherein the ocular tissue graft comprises a curved portion in the shape of a portion of a sphere.

31. (canceled)

32. A method of treating a disease or injury of the eye in a subject, the method comprising the step of contacting the eye of the subject with: (a) a tissue graft product comprising a plurality of layers of extracellular matrix laminated together, wherein the extracellular matrix is derived from an amniotic membrane, and wherein the stromal side of an extracellular matrix layer is presented on both the upper and lower surfaces of the tissue graft product: or (ii) an ocular tissue graft comprising a plurality of layers of extracellular matrix laminated together, wherein the extracellular matrix is derived from an amniotic membrane, and wherein the stromal side of an extracellular matrix layer is presented on both the upper and lower surfaces of the tissue graft product, wherein the ocular tissue graft comprises a curved portion in the shape of a portion of a sphere, so as thereby to treat the subject.

33. The method of claim 32, wherein the disease or injury of the eye in the subject comprises: (i) an abrasion: (ii) a chemical exposure: (iii) a cut or laceration; a disease or injury of the cornea: (iii) repair of a damaged tissue: (iv) a reduction in scar tissue; (v) a reduction in scar tissue formation relative to an untreated eye: (vi) increasing epithelial cell migration relative to an untreated eye: (vii) increasing epithelial cell adhesion relative to an untreated eye: (viii) increasing epithelial cell proliferation relative to an untreated eye: or (ix) increasing epithelial cell coverage relative to an untreated eye.

34-42. (canceled)

43. The method of claim 32, wherein the subject is a mammal.

44. The method of claim 43, wherein the subject is a human.
